SEQUENCE LISTING (12 sequences)

SEQ ID NO: 1 - protein, 107 aa, Homo sapiens [sequence omitted]

SEQ ID NO: 2 - protein, 214 aa, Homo sapiens
DIQMTQSPSSLSASVGDRVTITCGTSEDIINYLNWYQQKPGKAPKLLIYHTSRLQSGVPSRFSGSGSGTDFTLTISSLQPEDFATYYCQQGYTLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 121 aa, Homo sapiens [sequence omitted]

SEQ ID NO: 4 - protein, 451 aa, Homo sapiens
EVQLVQSGAEVKKPGASVKVSCKASGYTFTSYVIHWVRQRPGQGLAWMGYINPYNDGTKYNERFKGKVTITSDRSTSTVYMELSSLRSEDTAVYLCGREGIRYYGLLGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 400 aa, Homo sapiens
DLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICHLWIKLFPPIPAPKSNIKDLFVTTNYEKAGSSETEIEVICYIEKPGVETLEDSVF

SEQ ID NO: 6 - protein, 398 aa, Mus musculus
DLLNHKKFLLLPPVNFTIKATGLAQVLLHWDPNPDQEQRHVDLEYHVKINAPQEDEYDTRKTESKCVTPLHEGFAASVRTILKSSHTTLASSWVSAELKAPPGSPGTSVTNLTCTTHTVVSSHTHLRPYQVSLRCTWLVGKDAPEDTQYFLYYRFGVLTEKCQEYSRDALNRNTACWFPRTFINSKGFEQLAVHINGSSKRAAIKPFDQLFSPLAIDQVNPPRNVTVEIESNSLYIQWEKPLSAFPDHCFNYELKIYNTKNGHIQKEKLIANKFISKIDDVSTYSIQVRAAVSSPCRMPGRWGEWSQPIYVGKERKSLVEWHLIVLPTAACFVLLIFSLICRVCHLWTRLFPPVPAPKSNIKDLPVVTEYEKPSNETKIEVVHCVEEVGFEVMGNSTF

SEQ ID NO: 7 - protein, 5 aa, Homo sapiens
SYVIH

SEQ ID NO: 8 - protein, 17 aa, Homo sapiens
YINPYNDGTKYNERFKG

SEQ ID NO: 9 - protein, 12 aa, Homo sapiens
EGIRYYGLLGDY

SEQ ID NO: 10 - protein, 11 aa, Homo sapiens
GTSEDIINYLN

SEQ ID NO: 11 - protein, 7 aa, Homo sapiens
HTSRLQS

SEQ ID NO: 12 - protein, 9 aa, Homo sapiens
QQGYTLPYT

(19) United States
(12) Patent Application Publication
(10) Pub. No.: US 2024/0409626 A1
(43) Pub. Date: Dec. 12, 2024

(54) METHODS FOR TREATING CHRONIC OBSTRUCTIVE PULMONARY DISEASE IN AN ENHANCED PATIENT POPULATION USING BENRALIZUMAB

(71) Applicant: ASTRAZENECA AB, SODERTALJE (SE)

(72) Inventors: Paul NEWBOLD, Wilmington, DE (US); UBALDO MARTIN, Wilmington, DE (US); MARIA JISON, Wilmington, DE (US); PETER BARKER, Wilmington, DE (US)

(21) Appl. No.: 18/817,113

(22) Filed: Aug. 27, 2024

Related U.S. Application Data
(63) Continuation of application No. 18/406,653, filed on Jan. 8, 2024, now abandoned.
Application No. 18/406,653 is a continuation of application No. 16/875,159, filed on May 15, 2020, now abandoned.
(60) Provisional application No. 62/848,875, filed on May 16, 2019.

(51) Int. Cl. C07K 16/24; A61P 11/00

(52) U.S. Cl. CPC C07K 16/244; A61P 11/00

(57) ABSTRACT

Provided herein is are methods of treating Chronic Obstructive Pulmonary Disease (COPD) in a patient, comprising administering to the patient an effective amount of benralizumab or an antigen-binding fragment thereof.

### BACKGROUND

Chronic obstructive pulmonary disease (COPD) is a significant cause of morbidity and mortality worldwide. In contrast to other chronic diseases, COPD is increasing in prevalence and is projected to be the third-leading cause of death and disability worldwide by 2020. The costs to society for treating COPD are high, accounting for approximately 3.4% of the total health care budget of the European Union. In the United States, the direct and indirect costs of COPD are estimated to be more than $30 billion.

Approximately 30% of patients with COPD have elevated levels of eosinophils in the airway as measured by sputum induction or bronchoalveolar lavage. In COPD, the response to oral and inhaled corticosteroids (ICS) is related to the intensity of the airway eosinophilic inflammation, and a sputum eosinophilia count of greater than 3% has been demonstrated to be a good predictor of response to steroids in COPD.

A strategy in which increasing therapy with corticosteroids was used to control sputum eosinophilia greater than 3% in COPD resulted in a reduction in the frequency of severe COPD exacerbations requiring admission to a hospital when patients were stepped up to oral corticosteroid therapy. Standard therapy for acute exacerbations of COPD (AECOPD) includes treatment of inflammation with systemic corticosteroids, which are associated with a reduction in length of hospital stay and hastened recovery. Corticosteroids are responsible for early apoptosis of eosinophils and generally result in a reduction in eosinophilia. However, long-term therapy with corticosteroids is associated with significant side effects such as suppression of the hypothalamic-pituitary-adrenal axis and osteoporosis, and corticosteroids do not avert exacerbations in all eosinophilic COPD patients.

COPD patients with increased sputum eosinophil counts have been shown to have significant improvements in forced expiratory volume in 1 second (FEV₁) and quality of life-scores that were associated with decreased sputum eosinophil counts and eosinophil cationic protein (ECP) levels. Thus, therapies specifically targeted at eosinophils in COPD may have beneficial effects.

Benralizumab is a humanized, afucosylated monoclonal antibody (mAb) that specifically binds to the alpha chain of human interleukin-5 receptor alpha (IL-5Rα), which is expressed on eosinophils. It induces apoptosis of these cells via antibody-dependent cell cytotoxicity.

Thus, given the high unmet need of treating COPD without the corticosteroid-induced side effects and the fact that some patients with COPD have an eosinophilic component, the effect of benralizumab on COPD in adult subjects was examined.

### BRIEF SUMMARY

Methods of treating chronic obstructive pulmonary disease (COPD) in a human COPD patient are provided herein.

In certain aspects, described herein is a method of treating chronic obstructive pulmonary disease (COPD) in a human COPD patient comprising administering to a COPD patient a dose of 100 mg of benralizumab or an antigen-binding fragment thereof, wherein prior to administration, the patient: (a) has a blood eosinophil count of ≥300 eosinophils/μL; (b) has experienced ≥2 prior exacerbations in the year prior to administration; and (c) is on triple background therapy comprising inhaled corticosteroid (ICS), long-acting beta agonist (LABA), and long-acting muscarinic antagonist (LAMA).

In certain aspects, described herein a method of reducing the annual exacerbation rate of COPD comprises administering to a human COPD patient a dose of 100 mg of benralizumab or an antigen-binding fragment thereof, wherein prior to administration, the patient: (a) has a blood eosinophil count of ≥300 eosinophils/μL; (b) has experienced ≥2 prior exacerbations in the year prior to administration; and (c) is on triple background therapy comprising inhaled corticosteroid (ICS), long-acting beta agonist (LABA), and long-acting muscarinic antagonist (LAMA). In certain aspects, described herein a method of reducing the annual exacerbation rate of severe or very severe COPD comprises administering to a human COPD patient a dose of 100 mg of benralizumab or an antigen-binding fragment thereof, wherein prior to administration, the patient: (a) has a blood eosinophil count of ≥300 eosinophils/μL; (b) has experienced ≥2 prior exacerbations in the year prior to administration; and (c) is on triple background therapy comprising inhaled corticosteroid (ICS), long-acting beta agonist (LABA), and long-acting muscarinic antagonist (LAMA).

In certain aspects, described herein is a method of increasing forced expiratory volume in one second (FEV₁) in a human chronic obstructive pulmonary disease (COPD) patient comprising a dose of 100 mg of benralizumab or an antigen-binding fragment thereof, wherein prior to administration, the patient: (a) has a blood eosinophil count of ≥300 eosinophils/μL; (b) has experienced ≥2 prior exacerbations in the year prior to administration; and (c) is on triple background therapy comprising inhaled corticosteroid (ICS), long-acting beta agonist (LABA), and long-acting muscarinic antagonist (LAMA).

In certain aspects, described herein is a method of increasing forced vital capacity (FVC) in a human chronic obstructive pulmonary disease (COPD) patient comprising administering to the patient a dose of 100 mg of benralizumab or an antigen-binding fragment thereof, wherein prior to administration, the patient: (a) has a blood eosinophil count of ≥300 eosinophils/μL; (b) has experienced ≥2 prior exacerbations in the year prior to administration; and (c) is on triple background therapy comprising inhaled corticosteroid (ICS), long-acting beta agonist (LABA), and long-acting muscarinic antagonist (LAMA).

In some embodiments, the patient has a history of two or three or more exacerbations in the year prior to the administration of benralizumab or antigen-binding fragment thereof.

In some embodiments, the patient receiving benralizumab is concurrently being treated with triple background therapy, wherein the triple background therapy comprises inhaled corticosteroid (ICS), long-acting beta agonist (LABA), and long-acting muscarinic antagonist (LAMA).

In some embodiments, the patient receiving benralizumab is not currently being treated with triple background therapy, wherein the triple background therapy comprises inhaled corticosteroid (ICS), long-acting beta agonist (LABA), and long-acting muscarinic antagonist (LAMA).

In some embodiments, the patient has a blood eosinophil count of at least 300 eosinophils/μL prior to the administration. In some embodiments, the patient has a blood eosinophil count of 300-450 eosinophils/μL prior to the administration. In certain aspects, the patient has a blood eosinophil count of greater than 400 eosinophils/μL prior to the administration. In certain aspects, the patient has a blood eosinophil count of greater than 450 eosinophils/μL prior to the administration. In some aspects, the patient has a blood eosinophil count of greater than 500 eosinophils/μL prior to the administration.

In some embodiments, the patient has moderate, severe, or very severe COPD as defined by Global Initiative for Chronic Obstructive Lung Disease (GOLD). In some embodiments, the patient has severe COPD as defined by GOLD. In other embodiments, the patient has very severe COPD as defined by GOLD.

In some embodiments, the administration reduces the exacerbation rate of COPD. In some embodiments, the exacerbation rate is reduced by at least 20% (e.g., at least 20%, at least 30%, at least 40%, at least 50%, or at least 60%). In some embodiments, the exacerbation rate is reduced within a year from the first administration of benralizumab or antigen-binding fragment thereof. In some embodiments, the exacerbation rate is reduced within six months from the first administration of benralizumab or antigen-binding fragment thereof.

In some embodiments, the patient had a post-bronchodilator FEV₁ of ≤80% (e.g., ≤70% or ≤65%) of predicted normal value prior to the administration of benralizumab or an antigen-binding fragment thereof.

In some embodiments, the administration increases the patient's FEV₁. In certain aspects, the increased FEV₁ is a pre-bronchodilator FEV₁. In some embodiments, the pre-bronchodilator FEV₁ is increased by at least 10%. In some embodiments, the pre-bronchodilator FEV₁ is increased by about 12%. In some embodiments, the increased FEV₁ is a post-bronchodilator FEV₁. In some embodiments, the post-bronchodilator FEV₁ is increased by at least 5%. In certain aspects, the post-bronchodilator FEV₁ is increased by about 7%. In some embodiments, the pre-bronchodilator FEV₁ and the post-bronchodilator FEV₁ increase. In some embodiments, the FEV₁ is increased within a year from the first administration of the benralizumab or antigen-binding fragment thereof.

In some embodiments, the administration increases the patient's FVC. In certain aspects, the increased FVC is a pre-bronchodilator FVC. In some embodiments, the increased FVC is a post-bronchodilator FVC. In some embodiments, the pre-bronchodilator FVC and the post-bronchodilator FVC increase. In some embodiments, the FVC is increased by at least 3%. In some embodiments, the FVC is increased within a year from the first administration of the benralizumab or antigen-binding fragment thereof.

In some embodiments, the administration improves a COPD questionnaire score assessing COPD symptoms. In certain aspects, the COPD questionnaire is the COPD-Specific Saint George's Respiratory Questionnaire (SGRQ-C). In certain aspects, the patient's SGRQ-C (symptom) score decreases by at least 5. In certain aspects, the patient's SGRQ-C (symptom) score decreases by at least 7.

In some embodiments, the patient had an FEV₁/forced vital capacity (FVC) of <0.70 prior to the administration of the benralizumab or antigen-binding fragment thereof.

In some embodiments, at least two doses of the benralizumab or an antigen-binding fragment thereof are administered. In some embodiments, a first dose of benralizumab or an antigen-binding fragment thereof is administered at day zero and a second dose is administered at 4 weeks. In some embodiments,, at least one dose of the benralizumab or an antigen-binding fragment thereof is administered at an interval of 8 weeks after the previous dose. In some embodiments,, the benralizumab or an antigen-binding fragment thereof is administered with at least one four-week dosing interval and then with at least one eight-week dosing interval. In some embodiments,, the benralizumab or an antigen-binding fragment thereof is administered with three four-week dosing intervals and then at eight-week dosing intervals.

In some embodiments, the benralizumab or antigen-binding fragment thereof is administered once every four weeks to once every twelve weeks. In some embodiments, the benralizumab or antigen-binding fragment thereof is administered once every four weeks. In some embodiments, the benralizumab or antigen-binding fragment thereof is administered once every eight weeks. In some embodiments, the benralizumab or antigen-binding fragment thereof is administered once every four weeks for twelve weeks and then once every eight weeks.

In some embodiments, the administration is subcutaneous.

In certain aspects, described herein is a method of treating chronic obstructive pulmonary disease (COPD) in a human COPD patient, comprising administering to the patient a dose of 100 mg benralizumab or an antigen-binding fragment thereof, wherein prior to the administration the patient: (a) has a blood eosinophil count of ≥300 eosinophils/μL; (b) has experienced ≥3 prior exacerbations in the year prior to administration; and (c) is on triple background therapy comprising inhaled corticosteroid (ICS), long-acting beta agonist (LABA), and long-acting muscarinic antagonist (LAMA).

In certain aspects, described herein is a method of reducing the exacerbation rate of chronic obstructive pulmonary disease (COPD) in a human COPD patient comprising administering to the patient a dose of 100 mg benralizumab or an antigen-binding fragment thereof, wherein prior to the administration the patient: (a) has a blood eosinophil count of ≥300 eosinophils/μL; (b) has experienced ≥3 prior exacerbations in the year prior to administration; and (c) is on triple background therapy comprising inhaled corticosteroid (ICS), long-acting beta agonist (LABA), and long-acting muscarinic antagonist (LAMA).

In certain aspects, described herein is a method of increasing forced expiratory volume in one second (FEV₁) in a human chronic obstructive pulmonary disease (COPD) patient comprising to the patient a dose of 100 mg benralizumab or an antigen-binding fragment thereof, wherein prior to the administration the patient: (a) has a blood eosinophil count of ≥300 eosinophils/μL; (b) has experienced ≥3 prior exacerbations in the year prior to administration; and (c) is on triple background therapy comprising inhaled corticosteroid (ICS), long-acting beta agonist (LABA), and long-acting muscarinic antagonist (LAMA).

In certain aspects, described herein is a method of increasing forced vital capacity (FVC) in a human chronic obstructive pulmonary disease (COPD) patient comprising administering to the patient a dose of 100 mg benralizumab or an antigen-binding fragment thereof, wherein prior to the administration the patient: (a) has a blood eosinophil count of ≥300 eosinophils/μL; (b) has experienced ≥3 prior exacerbations in the year prior to administration; and (c) is on triple background therapy comprising an inhaled corticosteroid (ICS), a long-acting beta agonist (LABA) and a long-acting muscarinic antagonist (LAMA).

### BRIEF DESCRIPTION OF THE DRAWINGS/FIGURES

FIG. 1 shows the flow diagram of the study described in Examples 1 and 2.

FIGS. 2A-B are graphs showing the annual exacerbation (all=FIG. 2A and severe=FIG. 2B) rates for patients in the study described in Examples 1 and 2. The data show that 100 mg of benralizumab is more efficacious than 10 mg and 30 mg in reducing annual exacerbation rates for COPD, and is particularly efficacious in reducing severe COPD exacerbations over the 10 mg and 30 mg doses.

FIGS. 3A-B are graphs showing change from baseline in lung function (FEV₁) and SGRQ Scores for GALATHEA (FIG. 3A) and TERRANOVA (FIG. 3B). The data overall show that 100 mg of benralizumab demonstrated a better treatment effect in improving lung function (FEV₁) and SGRQ scores over the 10 mg and 30 mg doses.

FIGS. 4A-B are graphs showing median blood eosinophil count over time with benralizumab treatment for patients with baseline blood eosinophil counts ≥220/μL in GALATHEA (FIG. 4A) and in TERRANOVA (FIG. 4B).

FIGS. 5A-B are graphs showing the annual COPD exacerbation rate ratio for patients receiving 100 mg of benralizumab in TERRANOVA analyzed by prior exacerbations (FIG. 5A) and by background therapy (FIG. 5B) in patients with baseline blood eosinophil counts ≥220/μL. TERRANOVA showed a 23% reduction in annual exacerbation rate (AER) for patients with ≥3 exacerbations in the year prior to administration, compared to 2% for patients with ≤2 exacerbations in the year prior to administration. TERRANOVA also showed a 17% reduction in AER for patients receiving triple inhaled therapy, compared to 7% reduction in AER for the overall population.

FIGS. 6A-B are graphs showing the annual COPD exacerbation rate ratio for patients receiving 100 mg of benralizumab in GALATHEA, analyzed by prior exacerbations (FIG. 6A) and by background therapy (FIG. 6B) patients with baseline blood eosinophil counts ≥220/μL. GALATHEA showed a 39% reduction in annual exacerbation rate (AER) for patients with ≥3 exacerbations in the year prior to administration, compared to 2% for patients with ≤2 exacerbations in the year prior to administration, and an 18% reduction in AER for patients receiving triple inhaled therapy versus placebo.

FIGS. 7A-B shows the annual COPD exacerbation rate ratio, analyzed by baseline blood eosinophil levels for GALATHEA (FIG. 7A) and TERRANOVA (FIG. 7B). As a stratifying criterion alone, there was no apparent trend between increasing baseline blood eosinophil count and treatment effect.

FIGS. 8A-8D show the annualized overall exacerbation rate (ERR) and severe exacerbation rate (Severe ERR) for GALATHEA and TERRANOVA in patients with ≥300 eosinophils/μL and on triple background therapy (ICS/LABA/LAMA), analyzed by prior exacerbations in the year prior to administration—ERR in patients with ≤2 exacerbations (FIG. 8A); Severe ERR in patients with ≤2 exacerbations (FIG. 8B); ERR in patients with ≥3 exacerbations (FIG. 8C); and Severe ERR in patients with ≥3 exacerbations (FIG. 8D). The data show that patients with >300 eosinophils/μL, on triple background therapy (ICS/LABA/LAMA), and with ≤2 exacerbations in the year prior to administration experienced benefit from benralizumab therapy. Patients with ≥300 eosinophils/μL, on triple background therapy (ICS/LABA/LAMA), and with ≥3 exacerbations in the year prior to administration experienced the greatest benralizumab treatment effect, particularly at the 100 mg dose.

FIG. 9A-D shows the annual COPD exacerbation rate ratio analyzed by respiratory disease characteristics for GALATHEA (FIGS. 9A, 9C) and TERRANOVA (FIGS. 9B, 9D) at 30 mg and 100 mg doses in patients with baseline eosinophil counts ≥220/μL. The data show that

FIGS. 10A-B show the effect of benralizumab versus placebo on annual exacerbation rate (AER) in moderate/severe and severe patients.

FIG. 11 shows the flow diagram of the study described in Example 3.

### DETAILED DESCRIPTION

The present disclosure describes a new study population that was identified based on the findings from GALATHEA and TERRANOVA, the previous Phase 3 studies of benralizumab in patients with moderate to very severe COPD, as described in Examples 1 and 2. Subgroup analyses in GALATHEA and TERRANOVA consistently identified patients with a history of more frequent exacerbations and those receiving triple background therapy as most likely to respond to benralizumab 100 mg treatment. In particular, subgroup analyses resulted in the following:
Annualized rate of severe COPD exacerbation was reduced by >30% with benralizumab 100 mg compared to placebo.
Patients on triple (ICS/LABA/LAMA) background therapy (approximately 70% and 60% of patients in GALATHEA and TERRANOVA, respectively) had a consistent treatment effect on annual COPD exacerbation rate with benralizumab 100 mg in both studies. At benralizumab 100 mg, GALATHEA showed an 18% reduction in AER over placebo in patients receiving triple background therapy. At benralizumab 100 mg, TERRANOVA showed a 17% reduction in AER for patients receiving triple background therapy compared 7% reduction in AER for the overall population.
Patients with more frequent exacerbations in the year prior to administration of benralizumab (≥3 versus ≤2) had a greater treatment effect on annual exacerbation rate of COPD and annual exacerbation rate of severe COPD with benralizumab 100 mg. However, patients with ≤2 exacerbations in the year prior to administration still experienced benefit from benralizumab therapy.
No treatment effect was observed on annual exacerbation rate of COPD in patients with a baseline blood eosinophil count <220/μL.

Findings in these subpopulations were consistent across the studies and supported by pooled analyses. Taken together, these characteristics identify those patients most likely to benefit from benralizumab treatment.

It is to be noted that the term “a” or “an” entity refers to one or more of that entity; for example, “an anti-IL-5α antibody” is understood to represent one or more anti-IL-5α antibodies. As such, the terms “a” (or “an”), “one or more,” and “at least one” can be used interchangeably herein.

Provided herein are methods for treating Chronic Obstructive Pulmonary Disease (COPD). The methods provided include administering an effective amount of benralizumab or an antigen-binding fragment thereof.

Information regarding benralizumab (or fragments thereof) for use in the methods provided herein can be found, e.g., in U.S. Patent Application Publication No. US 2010/0291073 A1, the disclosure of which is incorporated herein by reference in its entirety. Benralizumab and antigen-binding fragments thereof for use in the methods provided herein comprise a heavy chain and a light chain or a heavy chain variable region and a light chain variable region. In a further aspect, benralizumab or an antigen-binding fragment thereof for use in the methods provided herein includes any one of the amino acid sequences of SEQ ID NOs: 1-4. In a specific aspect, benralizumab or an antigen-binding fragment thereof for use in the methods provided herein comprises a light chain variable region comprising the amino acid sequence of SEQ ID NO: 1 and a heavy chain variable region comprising the amino acid sequence of SEQ ID NO:3. In a specific aspect, benralizumab or an antigen-binding fragment thereof for use in the methods provided herein comprises a light chain comprising the amino acid sequence of SEQ ID NO: 2 and heavy chain comprising the amino acid sequence of SEQ ID NO:4. In a specific aspect, benralizumab or an antigen-binding fragment thereof for use in the methods provided herein comprises a heavy chain variable region and a light chain variable region, wherein the heavy chain variable region comprises the Kabat-defined CDR1, CDR2, and CDR3 sequences of SEQ ID NOs: 7-9, and wherein the light chain variable region comprises the Kabat-defined CDR1, CDR2, and CDR3 sequences of SEQ ID NOs: 10-12. Those of ordinary skill in the art would easily be able to identify Chothia-defined, Abm-defined or other CDRs. In a specific aspect, benralizumab or an antigen-binding fragment thereof for use in the methods provided herein comprises the variable heavy chain and variable light chain CDR sequences of the KM1259 antibody as disclosed in U.S. Pat. No. 6,018,032, which is herein incorporated by reference in its entirety.

An acute exacerbation of COPD (AECOPD) is a sustained worsening of a patient's condition from the stable state and beyond normal day-to-day variations that is acute in onset and necessitates a change in regular medication in a patient with underlying COPD.

In certain aspects, a patient presenting at a physician's office or emergency department (ED) with COPD is administered benralizumab or an antigen-binding fragment thereof. Given the ability of benralizumab to reduce or deplete eosinophil counts for up to 12 weeks or more (see US 2010/0291073), benralizumab or an antigen-binding fragment thereof can be administered only once or infrequently while still providing benefit to the patient. In further aspects, the patient is administered additional follow-on doses. Follow-on doses can be administered at various time intervals depending on the patient's age, weight, ability to comply with physician instructions, clinical assessment, eosinophil count (blood or sputum eosinophils or eosinophilic cationic protein (ECP) measurement), or and other factors, including the judgment of the attending physician. The intervals between doses can be every 4 weeks, every 5 weeks, every 6 weeks, every 8 weeks, every 10 weeks, every 12 weeks, or longer intervals. In certain aspects, the intervals between doses can be every 4 weeks or every 8 weeks. In certain aspects, the intervals between doses can be every 4 weeks and every 8 weeks. In certain aspects, benralizumab or an antigen-binding fragment thereof is administered with three four-week dosing intervals (i.e., on Day 0, Week 4, and Week 8) and then with eight-week dosing intervals (i.e., on Week 16, Week 24, Week 32, etc.). In certain aspects, benralizumab or an antigen-binding fragment thereof is administered once every 4 weeks for 12 weeks. In certain aspects, benralizumab or an antigen-binding fragment thereof is administered once every 4 weeks for 12 weeks and then once every 8 weeks.

In certain aspects, the single dose or first dose is administered to the COPD patient shortly after the patient presents with an acute exacerbation, e.g., a mild, moderate or severe exacerbation. For example, the single or first dose of benralizumab or an antigen-binding fragment thereof can be administered during the presenting clinic or hospital visit, or in the case of very severe exacerbations, within 1, 2, 3, 4, 5, 6, 7, or more days, e.g., 7 days of the acute exacerbation, allowing the patient's symptoms to stabilize prior to administration of benralizumab.

In some embodiments, at least two doses of benralizumab or antigen-binding fragment thereof are administered to the patient. In some embodiments, at least three doses, at least four doses, at least five doses, at least six doses, or at least seven doses are administered to the patient. In some embodiments, benralizumab or an antigen-binding fragment thereof is administered over the course of four weeks, over the course of eight weeks, over the course of twelve weeks, over the course of twenty-four weeks, over the course of forty-eight weeks, or over the course of a year or more.

The amount of benralizumab or an antigen-binding fragment thereof to be administered to the patient can depend on various parameters such as the patient's age, weight, clinical assessment, eosinophil count (blood or sputum eosinophils, eosinophilic cationic protein (ECP) measurement, or eosinophil derived neurotoxin (EDN) measurement), or and other factors, including the judgment of the attending physician. In certain aspects, the dosage or dosage interval is not dependent on the eosinophil level.

In certain aspects, the patient is administered one or more doses of benralizumab or an antigen-binding fragment thereof wherein the dose is about 100 mg.

In certain aspects, administration of benralizumab or an antigen-binding fragment thereof according to the methods provided herein is through parenteral administration. For example, benralizumab or an antigen-binding fragment thereof can be administered by intravenous infusion or by subcutaneous injection. In certain embodiments, benralizumab or an antigen-binding fragment thereof can be administered by subcutaneous injection.

In certain aspects, benralizumab or an antigen-binding fragment thereof is administered according to the methods provided herein in combination or in conjunction with additional therapies. Such therapies include, without limitation, corticosteroid therapy (including inhaled corticosteroids (ICS)), long-acting β agonists (LABA, including long-acting β2 agonists), long-acting muscarinic antagonist (LAMA, including tiotropium), or other standard therapies. In certain aspects, benralizumab or an antigen-binding fragment there of is administered according to the methods provided herein in combination or in conjunction with ICS and LABA, with LABA and LAMA, or with ICS, LABA, and LAMA. In certain aspects, the term “triple background therapy” means ICS, LABA, and LAMA.

In certain instances, administration of benralizumab or an antigen-binding fragment thereof decreases COPD exacerbations including, for example, as measured by an exacerbation rate, an annual exacerbation rate, time to first exacerbation, and/or an annual rate of COPD exacerbations that are associated with an emergency room visit or hospitalization.

The methods provided herein can reduce exacerbation rates in COPD patients. In certain aspects, use of the methods provided herein, i.e., administration of benralizumab or an antigen-binding fragment thereof reduces the number of exacerbations experienced by the patient as compared to the number of exacerbations expected according to the patient's history, as compared to the average number of exacerbations expected in a comparable population of patients, or as compared to a comparable population treated with placebo over the same time period. In certain aspects, administration of benralizumab or an antigen-binding fragment thereof reduces the number of exacerbations in COPD patients with eosinophil counts of at least 300 eosinophils/μL prior to the administration. In certain aspects, administration of benralizumab or an antigen-binding fragment thereof reduces the number of exacerbations in COPD patients with eosinophil counts of at least 400 eosinophils/μL prior to the administration. In certain aspects, administration of benralizumab or an antigen-binding fragment thereof reduces the number of exacerbations in COPD patients with severe COPD as defined by the Global Initiative for Chronic Obstructive Lung Disease (GOLD), Global strategy for the diagnosis, management, and prevention of chronic obstructive pulmonary disease (revised 2018). In certain aspects, administration of benralizumab or an antigen-binding fragment thereof reduces the number of exacerbations in COPD patients with very severe COPD as defined by the GOLD. In certain aspects, administration of benralizumab or an antigen-binding fragment thereof reduces the number of exacerbations in COPD patients with severe or very severe COPD as defined by the GOLD. In certain aspects, administration of benralizumab or an antigen-binding fragment thereof reduces the number of exacerbations in COPD patients who are receiving corticosteroids (e.g., inhaled corticosteroids (ICS), long-acting β-agonists (LABA) (e.g., long-acting β2-agonists), and long-acting muscarinic antagonists (LAMA) (e.g., tiotropium).

In certain aspects, administration of benralizumab or an antigen-binding fragment thereof reduces the number of exacerbations (e.g., reduces the annual exacerbation rate) of COPD in patients who have experienced ≥2 prior exacerbations in the year prior to administration. In certain aspects, administration of benralizumab or an antigen-binding fragment thereof reduces the number of exacerbations (e.g., reduces the annual exacerbation rate) of COPD in patients who have experienced ≥3 prior exacerbations in the year prior to administration. In certain aspects, administration of benralizumab or an antigen-binding fragment thereof reduces the number of severe COPD exacerbations (e.g., reduces the annual severe exacerbation rate) in patients who have experienced ≥3 prior exacerbations in the year prior to administration.

In certain aspects, administration of benralizumab or an antigen-binding fragment thereof reduces the number of exacerbations (e.g., reduces the annual exacerbation rate) of COPD in patients who exhibit the following characteristics prior to administration: a) has a blood eosinophil count of ≥300 eosinophils/μL; b) has experienced ≥2 prior exacerbations in the year prior to administration; and c) is on triple background therapy comprising inhaled corticosteroids (ICS), long-acting β-agonists (LABA) (e.g., long-acting β2-agonists), and long-acting muscarinic antagonists (LAMA) (e.g., tiotropium). In certain aspects, administration of benralizumab or an antigen-binding fragment thereof reduces the number of exacerbations (e.g., reduces the annual exacerbation rate) of COPD in patients who exhibit the following characteristics prior to administration: a) has a blood eosinophil count of ≥300 eosinophils/μL; b) has experienced ≥3 prior exacerbations in the year prior to administration; and c) is on triple background therapy comprising inhaled corticosteroids (ICS), long-acting β-agonists (LABA) (e.g., long-acting β2-agonists), and long-acting muscarinic antagonists (LAMA) (e.g., tiotropium).

In certain aspects, administration of benralizumab or an antigen-binding fragment thereof treats chronic obstructive pulmonary disease (COPD) in patients who exhibit the following characteristics prior to administration: (a) has a blood eosinophil count of ≥300 eosinophils/μL; (b) has experienced ≥3 prior exacerbations in the year prior to administration; and (c) is on triple background therapy comprising inhaled corticosteroid (ICS), long-acting beta agonist (LABA), and long-acting muscarinic antagonist (LAMA); wherein the patient no longer receives triple background therapy after administering 100 mg of benralizumab once every four weeks for twelve weeks and then once every eight weeks. In certain aspects, administration of benralizumab or an antigen-binding fragment thereof treats chronic obstructive pulmonary disease (COPD) in patients who exhibit the following characteristics prior to administration: (a) has a blood eosinophil count of ≥300 eosinophils/μL; (b) has experienced ≥2 prior exacerbations in the year prior to administration; and (c) is on triple background therapy comprising inhaled corticosteroid (ICS), long-acting beta agonist (LABA), and long-acting muscarinic antagonist (LAMA); wherein the patient no longer receives triple background therapy after administering 100 mg of benralizumab once every four weeks for twelve weeks and then once every eight weeks,

In certain aspects, administration of benralizumab or an antigen-binding fragment thereof reduces exacerbations by at least about 15%, by at least about 20%, by at least about 25%, by at least about 30%, at least about 35%, at least about 40%, at least about 45%, at least about 50%, or at least about 55%, or at least 60%. In some embodiments, exacerbations are reduced about 17%, or about 18%. In some embodiments, administration of benralizumab or an antigen-binding fragment thereof reduces severe exacerbations by at least about 15%, by at least about 20%, by at least about 25%, by at least about 30%, at least about 35%, at least about 40%, at least about 45%, at least about 50%, or at least about 55%, or at least 60%. In some embodiments, severe exacerbations are reduced about 32%, or about 43%. The exacerbations (moderate or severe) can be reduced, for example, within a year from the first administration of benralizumab or antigen-binding fragment thereof. In an aspect, the exacerbations (moderate or severe) can be reduced within 6 months from the first administration of benralizumab or antigen-binding fragment thereof.

In certain aspects, use of the methods provided herein, i.e., administration of benralizumab or an antigen-binding fragment thereof, reduces rate of exacerbation (e.g., reduces annual rate of exacerbation) in COPD (e.g., moderate or severe) within 4 weeks, within 8 weeks, within 12 weeks, within 16 weeks, within 20 weeks, within 24 weeks, within 28 weeks, within 32 weeks, within 36 weeks, within 40 weeks, within 44 weeks, within 48 weeks, or within 52 weeks.

The methods provided herein can also reduce rate of exacerbation (e.g., reduce annual rate of exacerbation) in COPD (e.g., moderate or severe) in patients with eosinophil counts of ≥300 eosinophils/μL prior to the administration, for example by at least 20%, at least 30%, at least 50%, or at least 60%.

The methods provided herein can also reduce rate or exacerbation (e.g., reduce annual rate of exacerbation) in COPD patients with severe or very severe COPD (as defined by GOLD), for example by at least 40%, at least 50%, at least 60%, or at least 70%.

As used herein, the terms “annual exacerbation rates,” “annualized exacerbation rate,” “rate of exacerbation,” or “rate of annual exacerbation” are used interchangeably. The methods provided herein can reduce annual exacerbation rates in moderate to very severe COPD patients. In assessing annual COPD exacerbation rates, a COPD exacerbation is defined as symptomatic worsening of COPD requiring:

Use of systemic corticosteroids for at least 3 days (a single depot injectable dose of corticosteroids is considered equivalent to a 3-day course of systemic corticosteroids; and/or

Use of antibiotics; and/or

An inpatient hospitalization due to COPD.

The methods provided herein can reduce the time to a first COPD exacerbation after a first administration of benralizumab or an antigen-binding fragment thereof as compared to after a first administration of placebo.

In some instances, administration of benralizumab or an antigen-binding fragment thereof decreases the likelihood of a COPD exacerbation (e.g., within 52 weeks of a first administration of benralizumab or an antigen-binding fragment thereof) as compared to the likelihood of a COPD exacerbation after treatment with placebo.

In some instances, administration of benralizumab or an antigen-binding fragment thereof decreases the annual rate of COPD exacerbations that are associated with an emergency room or hospitalization as compared to administration of placebo.

In certain instances, administration of benralizumab or an antigen-binding fragment thereof improves the pulmonary function in a COPD patient, for example, as measured by forced expiratory volume in one second (FEV₁) or forced vital capacity.

The methods provided herein can increase forced expiratory volume in one second (FEV₁) in COPD patients. An increase can be measured based on the expected FEV₁ based on a large patient population, on the FEV₁ measured in a control population, or on the individual patient's FEV₁ prior to administration. In certain aspects, use of the methods provided herein, i.e., administration of benralizumab or an antigen-binding fragment thereof, can increase the FEV₁, as compared to the patient's baseline FEV₁. In some embodiments, the increased FEV₁ is pre-bronchodilator FEV₁. In some embodiments, the increased FEV₁ is post-bronchodilator FEV₁. In some embodiments, the increased FEV₁ is pre-bronchodilator FEV₁ and post-bronchodilator FEV₁. The FEV₁ (e.g., the pre-bronchodilator and/or post-bronchodilator FEV₁) can be increased, for example, within a year from the first administration of benralizumab or the antigen-binding fragment thereof.

A “bronchodilator,” as used herein, refers to any drug that widens or dilates the bronchi and bronchioles or air passages of the lungs, decreases resistance in the respiratory airway, and/or eases breathing by relaxing bronchial smooth muscle. For example, bronchodilators include short- and long-acting β2-agonists such as albuterol/salbutamol and other drugs commonly used to treat asthma.

In some embodiments, the patient had a post-bronchodilator FEV1 of ≤80% (e.g., ≤70% or ≤65%) of predicted normal value prior to the administration of benralizumab or an antigen-binding fragment thereof.

In certain aspects, the methods provided herein can increase FEV₁ by at least 5% or by at least 10%. In certain aspects, the methods provided herein can increase FEV₁ by about 12%. In certain aspects, the methods provided herein can increase pre-bronchodilator FEV₁ by at least 5% or by at least 10%. In certain aspects, the methods provided herein can increase pre-bronchodilator FEV₁ by about 12%.

In certain aspects, the methods provided herein can increase FEV₁ by at least 5%. In certain aspects, the methods provided herein can increase FEV₁ by about 7%. In certain aspects, the methods provided herein can increase post-bronchodilator FEV₁ by at least 5%. In certain aspects, the methods provided herein can increase post-bronchodilator FEV₁ by about 7%.

In certain aspects, the methods provided herein can increase pre-bronchodilator and post-bronchodilator FEV₁ by at least 5%. In certain aspects, the methods provided herein can increase can increase pre-bronchodilator by at least 10% and post-bronchodilator FEV₁ by at least 5%. In certain aspects, the methods provided herein can increase pre-bronchodilator FEV₁ by about 12% and post-bronchodilator FEV₁ by about 7%.

As provided herein, administration of benralizumab or the antigen-binding fragment thereof can also increase the percent predicted FEV₁ in COPD patients e.g., pre-bronchodilator and/or post-bronchodilator. By way of example, the percent predicted FEV₁ can increase by about 3.0, about 3.5, about 4.0, or about 4.5.

The methods provided herein can increase FEV₁ in COPD patients with blood eosinophil counts of at least 300 eosinophils/μL, and/or in patients receiving corticosteroids (e.g., inhaled corticosteroids (ICS), long-acting β-agonists (LABA) (e.g., long-acting β2-agonists), and long-acting muscarinic antagonists (e.g., tiotropium), and/or in patients who have experienced ≥2 or ≥3 prior exacerbations in the year prior to administration. In certain aspects, the methods provided herein can increase FEV1 in COPD patients: a) with blood eosinophil counts of at least 300 eosinophils/μL, b) receiving corticosteroids (e.g., inhaled corticosteroids (ICS), long-acting β-agonists (LABA) (e.g., long-acting β2-agonists), and long-acting muscarinic antagonists (e.g., tiotropium), and c) who have experienced ≥2 prior exacerbations in the year prior to administration. In certain aspects, the methods provided herein can increase FEV1 in COPD patients: a) with blood eosinophil counts of at least 300 eosinophils/μL, b) receiving corticosteroids (e.g., inhaled corticosteroids (ICS), long-acting β-agonists (LABA) (e.g., long-acting β2-agonists), and long-acting muscarinic antagonists (e.g., tiotropium), and c) who have experienced ≥3 prior exacerbations in the year prior to administration. In certain aspects, the methods provided herein can increase FEV₁ in such patients by at least 10% or by at least 15%. In certain aspects, the methods provided herein can increase pre-bronchodilator FEV₁ in such patients by at least 10% or by at least 15%. In certain aspects, the methods provided herein can increase post-bronchodilator FEV₁ in such patients by about 10%. In certain aspects, the methods provided herein can increase pre-bronchodilator FEV₁ and post-bronchodilator FEV₁ in such patients by at least 10%. In certain aspects, the methods provided herein can increase pre-bronchodilator FEV₁ in such patients by at least 15% and post-bronchodilator FEV₁ in such patients by at least 10%.

The methods provided herein can increase FEV₁ in COPD patients with blood eosinophil counts of at least 300 eosinophils/μL or in COPD patients with severe or very severe COPD as defined by the Global Initiative for Chronic Obstructive Lung Disease (GOLD). In certain aspects, the methods provided herein can increase FEV₁ in such patients by at least 15% or by at least 20%. In certain aspects, the methods provided herein can increase pre-bronchodilator FEV₁ in such patients by at least 15% or by at least 20%. In certain aspects, the methods provided herein can increase post-bronchodilator FEV₁ in such patients by about 15%. In certain aspects, the methods provided herein can increase pre-bronchodilator FEV₁ and post-bronchodilator FEV₁ in such patients by at least 15%. In certain aspects, the methods provided herein can increase pre-bronchodilator FEV₁ in such patients by at least 20% and post-bronchodilator FEV₁ in such patients by at least 15%.

In certain aspects, use of the methods provided herein, i.e., administration of benralizumab or an antigen-binding fragment thereof, increases the FEV₁ within 4 weeks, within 8 weeks, within 12 weeks, within 16 weeks, within 20 weeks, within 24 weeks, within 28 weeks, within 32 weeks, within 36 weeks, within 40 weeks, within 44 weeks, within 48 weeks, within 52weeks, or within 56 weeks or more. In certain aspects, administration of benralizumab or an antigen-binding fragment thereof improves FEV₁ within 52 weeks of a first administration of the benralizumab or antigen-binding fragment thereof. Use of the methods provided herein can increase FEV₁ by at least 0.05 L, at least 0.1 L, at least 0.13 L, at least 0.15 L, at least 0.20 L, at least 0.21 L, at least 0.22 L, at least 0.23 L, at least 0.24 L, or at least 0.25 L, at least 0.30 L, at least 0.35 L, at least 0.40 L, at least 0.45 L, or at least 0.50 L over the 56-week period.

The methods provided herein can increase forced vital capacity (FVC) in COPD patients. An increase can be measured based on the expected FVC based on a large patient population, on the FVC measured in a control population, or on the individual patient's FVC prior to administration. In certain aspects, use of the methods provided herein, i.e., administration of benralizumab or an antigen-binding fragment thereof, can increase the FVC, as compared to the patient's baseline FVC. In some embodiments, the increased FVC is pre-bronchodilator FVC. In some embodiments, the increased FVC is post-bronchodilator FVC. In some embodiments, the increased FVC is pre-bronchodilator FVC and post-bronchodilator FVC. The FVC (e.g., the pre-bronchodilator and/or post-bronchodilator FVC) can be increased, for example, within a year from the first administration of benralizumab or the antigen-binding fragment thereof.

In certain aspects, the methods provided herein can increase FVC by at least 3%. In certain aspects, the methods provided herein can increase pre-bronchodilator FVC by at least 2%, at least 3%., at least 5% or at least 10%. In certain aspects, the methods provided herein can increase post-bronchodilator FVC by at least 2%, at least 3%, at least 5% or at least 10%. In certain aspects, the methods provided herein can increase pre-bronchodilator and post-bronchodilator FVC by at least 2%, at least 3%., at least 5% or at least 10%. In certain aspects, use of the methods provided herein, i.e., administration of benralizumab or an antigen-binding fragment thereof, increases FVC within 4 weeks, within 8 weeks, within 12 weeks, within 16 weeks, within 20 weeks, within 24 weeks, within 28 weeks, within 32 weeks, within 36 weeks, within 40 weeks, within 44 weeks, within 48 weeks, within 52 weeks, or within 56 weeks or more.

In certain instances, administration of benralizumab or an antigen-binding fragment thereof improves respiratory symptoms in a COPD patient, for example, as measured by the Baseline/Transitional Dyspnea Index (BDI/TDI) and/or the Exacerbations of Chronic Pulmonary Disease Tool—Respiratory Symptoms (E-RS).

Provided herein are also methods for improving respiratory symptoms as measured by the Baseline/Transitional Dyspnea Index (TDI). For example, administration of benralizumab or an antigen-binding fragment thereof can improve (increase) a COPD patient's BDI score by at least 1, at least 2, or at least 3 and/or result in a positive TDI score. The BDI/TDI score can be improved, for example, within a year from the first administration of benralizumab or the antigen-binding fragment thereof.

In certain aspects, use of the methods provided herein, i.e., administration of benralizumab or an antigen-binding fragment thereof, improves a BDI/TDI score within 4 weeks, within 8 weeks, within 12 weeks, within 16 weeks, within 20 weeks, within 24 weeks, within 28 weeks, within 32 weeks, within 36 weeks, within 40 weeks, within 44 weeks, within 48 weeks, within 52 weeks, or within 56 weeks or more.

Provided herein are also methods for improving respiratory symptoms as measured by the Exacerbations of Chronic Pulmonary Disease Tool—Respiratory Symptoms (E-RS). For example, administration of benralizumab or an antigen-binding fragment thereof can improve (decrease) a COPD patient's E-RS score by least 3, at least 4, at least 6, at least 7, at least 8, at least 9, or at least 10. The E-RS score can be improved, for example, within a year from the first administration of benralizumab or the antigen-binding fragment thereof.

In certain aspects, use of the methods provided herein, i.e., administration of benralizumab or an antigen-binding fragment thereof, improves a E-RS score within 4 weeks, within 8 weeks, within 12 weeks, within 16 weeks, within 20 weeks, within 24 weeks, within 28 weeks, within 32 weeks, within 36 weeks, within 40 weeks, within 44 weeks, within 48 weeks, within 52 weeks, or within 56 weeks or more.

In certain instances, administration of benralizumab or an antigen-binding fragment thereof improves the health status and/or health-related quality of life in a COPD patient, for example, as measured by the Saint George's Respiratory Questionnaire (SGRQ), the COPD-Specific Saint George's Respiratory Questionnaire (SGRQ-C), and/or the COPD assessment tool (CAT).

Provided herein are methods for improving COPD symptoms, e.g., as assessed using a COPD questionnaire such as the Saint George's Respiratory Questionnaire (SGRQ). For example, administration of benralizumab or an antigen-binding fragment thereof can improve a patient's SGRQ score by at least 2, at least 3, at least 4, at least 6, at least 7, at least 8, at least 9, or at least 10. The SGRQ score can be improved, for example, within a year from the first administration of benralizumab or the antigen-binding fragment thereof.

In certain aspects, use of the methods provided herein, i.e., administration of benralizumab or an antigen-binding fragment thereof, improves a SGRQ score within 4 weeks, within 8 weeks, within 12 weeks, within 16 weeks, within 20 weeks, within 24 weeks, within 28 weeks, within 32 weeks, within 36 weeks, within 40 weeks, within 44 weeks, within 48 weeks, within 52 weeks, or within 56 weeks or more. In certain aspects, administration of benralizumab or an antigen-binding fragment thereof improves an SGRQ score within 52 weeks of a first administration of the benralizumab or antigen-binding fragment thereof.

Provided herein are also methods for improving COPD symptoms, e.g., as assessed using a COPD questionnaire such as the COPD-Specific Saint George's Respiratory Questionnaire (SGRQ-C). For example, administration of benralizumab or an antigen-binding fragment thereof can improve a COPD patient's SGRQ-C (symptom) score by at least 2, at least 3, at least 4, at least 6, at least 7, at least 8, at least 9, or at least 10. The SGRQ-C (symptom) score can be improved, for example, within a year from the first administration of benralizumab or the antigen-binding fragment thereof.

In certain aspects, use of the methods provided herein, i.e., administration of benralizumab or an antigen-binding fragment thereof, improves a SGRQ-C (symptom) score within 4 weeks, within 8 weeks, within 12 weeks, within 16 weeks, within 20 weeks, within 24 weeks, within 28 weeks, within 32 weeks, within 36 weeks, within 40 weeks, within 44 weeks, within 48 weeks, within 52 weeks, or within 56 weeks or more.

Provided herein are also methods for improving COPD symptoms, e.g., as assessed using the COPD assessment tool (CAT). For example, administration of benralizumab or an antigen-binding fragment thereof can improve (decrease) a COPD patient's CAT score by at least 2, at least 3, at least 4, at least 6, at least 7, at least 8, at least 9, or at least 10. The CAT score can be improved (decreased), for example, within a year from the first administration of benralizumab or the antigen-binding fragment thereof.

In certain aspects, use of the methods provided herein, i.e., administration of benralizumab or an antigen-binding fragment thereof, improves (decreases) a CAT score within 4 weeks, within 8 weeks, within 12 weeks, within 16 weeks, within 20 weeks, within 24 weeks, within 28 weeks, within 32 weeks, within 36 weeks, within 40 weeks, within 44 weeks, within 48 weeks, within 52 weeks, or within 56 weeks or more.

In certain aspects, use of the methods provided herein, i.e., administration of benralizumab or an antigen-binding fragment thereof, reduces nocturnal awakenings.

In certain aspects, use of the methods provided herein, i.e., administration of benralizumab or an antigen-binding fragment thereof, reduces the use of rescue medication.

In certain aspects, use of the methods provided herein, i.e., administration of benralizumab or an antigen-binding fragment thereof, reduces the severity, frequency, and/or duration of EXACT-PRO defined events.

In certain aspects, use of the methods provided herein, i.e., administration of benralizumab or an antigen-binding fragment thereof, reduces the annual rate of hospitalizations due to COPD.

In certain aspects, use of the methods provided herein, i.e., administration of benralizumab or an antigen-binding fragment thereof, reduces the annual rate of hospitalizations and emergency department visits due to COPD.

In certain aspects, use of the methods provided herein, i.e., administration of benralizumab or an antigen-binding fragment thereof, reduces the annual rate of unscheduled outpatient visits due to COPD.

In certain aspects, use of the methods provided herein, i.e., administration of benralizumab or an antigen-binding fragment thereof, reduces the annual rate of unscheduled healthcare encounters due to COPD.

In certain aspects, use of the methods provided herein, i.e., administration of benralizumab or an antigen-binding fragment thereof, reduces COPD-specific resource utilization. For example, administration of benralizumab or an antigen-binding fragment thereof can reduce unscheduled physician visits, unscheduled phone calls to physicians, and/or use of other COPD medications.

In certain aspects, use of the methods provided herein, i.e., administration of benralizumab or an antigen-binding fragment thereof, reduces the annualized rate of severe COPD exacerbations, where a severe COPD exacerbation is defined by symptomatic worsening of COPD requiring an inpatient hospitalization or results in death due to COPD.

In certain aspects, use of the methods provided herein, i.e., administration of benralizumab or antigen-binding fragment thereof to a COPD patient, increases forced expiratory volume in one second (FEV₁), increases forced vital capacity (FVC), reduces COPD exacerbation rate, and/or improves a COPD questionnaire score (e.g., the COPD control questionnaire).

In certain aspects, use of the methods provided herein, i.e., administration of benralizumab or antigen-binding fragment thereof to a COPD patient, decreases annual COPD exacerbation rate, improves SGRQ scores, and increases FEV₁ (e.g., in COPD patients with a baseline blood eosinophil count ≥300 cells/μL).

In certain aspects, the COPD patient was prescribed or has been using corticosteroids (e.g., inhaled corticosteroids (ICS)), long-acting β-agonists (LABA, e.g., long-acting β2-agonists) and a long-acting muscarinic antagonist (LAMA) prior to administration of benralizumab or antigen-binding fragment thereof. In certain aspects, the COPD patient is treated with ICS, LABA, and LAMA concurrently with benralizumab or antigen-binding fragment thereof. In certain aspects, the COPD patient is not concurrently treated with ICS, LABA, and LAMA and benralizumab or antigen-binding fragment thereof.

In certain aspects of the methods provided herein, the patient has a history of COPD exacerbations. In certain aspects, the history of exacerbations comprises at least two exacerbations in the year prior to the administration of benralizumab or an antigen-binding fragment thereof. In certain aspects, the history of exacerbations comprises at least three exacerbations in the year prior to the administration of benralizumab or an antigen-binding fragment thereof. In certain aspects, the patient has a forced expiratory volume (FEV₁) of less than 80% predicted value prior to the administration. In certain aspects, the patient has an FEV₁/FVC of less than 0.70 prior to the administration.

In certain aspects, the COPD patient has a particular blood eosinophil count, e.g., prior to the administration of benralizumab or an antigen-binding fragment thereof. Blood eosinophil counts can be measured, for example, using a complete blood count (CBC) with cell differential. In certain aspects, the COPD patient has a blood eosinophil count of ≥300 eosinophils/μL prior to the administration of benralizumab or an antigen-binding fragment thereof. In certain aspects, the patient has a blood eosinophil count of 300-450 eosinophils/μL prior to the administration of benralizumab or an antigen-binding fragment thereof. In certain aspects, the patient has a blood eosinophil count of greater than 400 eosinophils/μL prior to the administration of benralizumab or an antigen-binding fragment thereof. In certain aspects, the patient has a blood eosinophil count of greater than 450 eosinophils/μL prior to the administration of benralizumab or an antigen-binding fragment thereof.

In certain aspects, the COPD patient has moderate COPD has defined by Global Initiative for Chronic Obstructive Lung Disease (GOLD), i.e., GOLD II. In certain aspects, the COPD patient has severe COPD has defined by Global Initiative for Chronic Obstructive Lung Disease (GOLD), i.e., GOLD III. In certain aspects, the COPD patients have very severe COPD as defined by GOLD, i.e., GOLD IV. In certain aspects, the COPD patient has severe or very severe COPD as defined by GOLD, i.e., GOLD III or IV.

### EXAMPLES

### Example 1: Patients and Methods

GALATHEA and TERRANOVA were Phase III complementary studies designed to evaluate efficacy (based on decrease in annual rate of moderate to severe exacerbations) and safety of benralizumab for patients with moderate to very severe COPD, at risk of exacerbations, and with blood eosinophil counts >220/μL (a threshold predicted to select for patients likely to respond to benralizumab, based on previous results). The following Examples report the primary results of GALATHEA and TERRANOVA.

### Trial Design

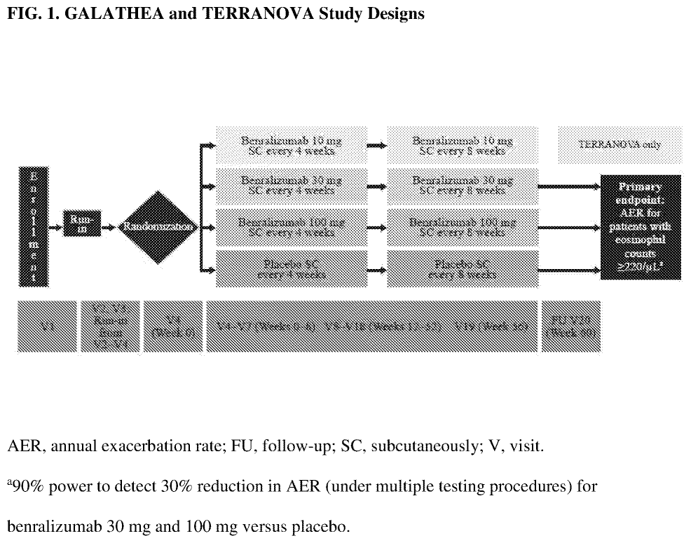

GALATHEA (NCT02138916) and TERRANOVA (NCT02155660) were Phase III, randomized, double-blind, placebo-controlled, parallel-group trials. Each trial comprised an enrollment visit, 3-week screening period, 56-week randomized treatment period, and follow-up visit (FIG. 1).

Eligible patients were stratified by country and blood eosinophil count (≥300/μL and <300/μL). Recruitment was capped centrally for cohorts with baseline eosinophil counts <220/μL, 220-299/μL, and ≥300/μL to maintain predefined cohort sizes and ˜2:1 ratio of patients with eosinophil counts ≥220/μL (primary analysis population) and <220/μL. This design enriched the trial population with patients likely to respond to benralizumab (blood eosinophil counts ≥220/μL) and allowed assessment of the benralizumab benefit: risk ratio for patients with COPD across a range of blood eosinophil counts. Dosage selection was based on approved asthma dose of 30 mg, 100 mg to inform safety margin, and 10 mg to evaluate dose-efficacy relationship.

At randomization, eligible patients were allocated in a 1:1:1 or 1:1:1:1 ratio, respectively, to receive placebo or benralizumab (30 mg or 100 mg in GALATHEA; 10 mg, 30 mg, or 100 mg in TERRANOVA). Full eligibility criteria are provided in the trial protocols (available at NEJM.org). Study drug was administered by subcutaneous injection every 4 weeks for the first three doses and every 8 weeks thereafter. Patients' maintenance and rescue medication use was recorded daily via electronic diary (eDiary) during the study. Eligibility for randomization included ≥70% compliance with prescribed inhaled maintenance therapy during the run-in period (based on eDiary).

The primary population for all analyses included patients with baseline blood eosinophil counts ≥220/μL. Primary end point for both trials was annualized COPD exacerbation rate at Week 56. Key secondary end points included change from baseline in prebronchodilator forced expiratory volume in 1 second (FEV₁) and St. George's Respiratory Questionnaire (SGRQ) score. Safety end points included types and frequencies of adverse events (AEs).

A COPD exacerbation was defined as symptomatic worsening of COPD resulting in use of oral corticosteroids for ≥3 days and/or use of antibiotics and/or hospitalization or death related to COPD (see Supplementary Appendix). Respiratory symptoms were recorded daily in an eDiary and assessed at center visits. Patients completed the SGRQ during center visits. Additional assessments utilized, but not reported, included the COPD Assessment Test, Baseline/Transitional Dyspnea Index, Exacerbations of Chronic Pulmonary Disease Tool—Patient-Reported Outcome, and EuroQOL 5D 5L. Spirometry data were collected at center visits, along with safety data, but reading of results was centralized (see Supplementary Appendix).

### Study Procedures

Data were collected from all patients at the following periods:
Enrollment (Week −4)
Every 2 weeks during run-in/screening period (Weeks −3 to −1)
Randomization (Week 0) and during the induction phase (Week 2)
Every 4 weeks during the reduction and maintenance phases (Weeks 4-56)
Follow up (Week 60)

### Spirometry

Patients received prebronchodilator spirometry at enrollment, at Visit 2 (Week 3) during run-in/screening phase, and at Weeks −3, 0, 4, 8, 16, 24, 32, 40, 48, and 56 during the treatment period. Patients received postbronchodilator spirometry at Visit 2 (Week −3), and a subset of patients also received postbronchodilator spirometry at enrollment, at Weeks −3, 0, 4, 8, 16, 24, 32, 40, 48, and 56. Spirometry was conducted by study investigators or authorized delegates according to American Thoracic Society/European Respiratory Society guidelines.

### COPD Assessment Test

The COPD Assessment Test (CAT) is an eight-item patient-reported outcome (PRO) tool developed to measure the impact of COPD on health status. The instrument uses semantic differential six-point response scales that are defined by contrasting adjectives to capture the impact of COPD. Content includes items related to cough, phlegm, chest tightness, breathlessness going up hills/stairs, activity limitation at home, confidence leaving home, sleep, and energy. A CAT total score is the sum of item responses. Scores range from 0-40, with higher scores indicative of greater COPD impact on health status. The CAT test was completed by patients at the following study center visits: Visit 4 (Week 0), Visit 6 (Week 4), Visit 7 (Week 8), Visit 9 (Week 16), Visit 11 (Week 24), Visit 13 (Week 32), Visit 15 (Week 40), Visit 17 (Week 48), and Visit 19 (Week 56).

### Assessment of COPD Exacerbations

A COPD exacerbation was defined as a change in the patient's usual COPD symptoms that lasted 2 or more days, was beyond normal day-to-day variation, was acute in onset, and may warrant a change in regular medication and lead to any of the following:
Use of systemic corticosteroids for at least 3 days
A single depot injectable dose of corticosteroids considered equivalent to a 3-day course of systemic corticosteroids
Use of antibiotics
An inpatient hospitalization due to COPD (defined as an inpatient admission ≥24 hours in the hospital, observation area, emergency department, or other equivalent health care facility, depending on the country and health care system)

COPD exacerbations were considered moderate if they required treatment with systemic steroids and/or antibiotics and did not result in hospitalization or death. Exacerbations were considered severe if they resulted in hospitalization or death. Symptoms were assessed by the patient each morning via an eDiary for the purposes of a symptom worsening alert. The purpose of this alert was to notify both the patient and the study center of a potential symptom worsening event that warrants contact between the patient and the center for further evaluation. Each morning the patient completed three questions pertaining to the major symptoms of a worsening event (dyspnea, sputum volume, and sputum color). Patient-reported worsening of one or more of these symptoms triggered assessment of the minor symptoms of a worsening event (sore throat, cold, fever without other cause, cough, and wheeze). Questions pertaining to the severity of symptoms versus their usual state had three response options (e.g., How breathless have you been in the last 24 hours? Less breathlessness than usual, usual level of breathlessness, more breathless than usual). Questions related to the presence or absence of a symptom had a dichotomous response (e.g., Have you had a sore throat in the last 24 hours? No, yes I had a sore throat).

If there was worsening of at least two major symptoms or one major and one minor symptom for 2 consecutive days an alert directed to the patient and site was generated by eDiary system. If an event was not associated with an eDiary symptom worsening alert as described above (e.g., technical issue, patient self-reports symptom worsening/exacerbation event, the exacerbation is identified during a visit or phone contact, the exacerbation is evaluated and treated at a non-study center, or an acute/severe symptom deterioration that is not captured in the ePRO system occurs), the investigator interviewed the patient and evaluated potential worsening and duration of the following symptoms: shortness of breath, mucus volume, mucus purulence, cough, wheezing, sore throat, cold symptoms such as a runny nose or nasal congestion, fever, and chest tightness, among other findings.

The start of an exacerbation was defined as the start date of systemic corticosteroids or antibiotic treatment or hospital admission, whichever occurred earlier, and the end date was defined as the last day of systemic corticosteroids or antibiotic treatment or hospital discharge, whichever occurred later. A COPD exacerbation that occurred ≤7 days of the last dose of systemic steroids (oral, intramuscular, intravenous) or antibiotics or the last day of hospitalization was counted as the same exacerbation event.

### Assessment of Blood Eosinophils

Patients were stratified by the absolute blood eosinophil count as assessed by a central laboratory (hematology sample taken at Visit 1 [Week-4]). Randomization capped at the study level for the baseline eosinophil cohorts (<220/μL, 220-299/μL, ≥300/μL) to keep predefined cohort sample size and approximately 2:1 ratio of patients above and below the boundary of 220/μL. Once any of the eosinophil cohorts were filled, patients allocated to that particular cohort based on the Visit 1 eosinophil count were screen failed.

### Safety

An independent data monitoring board evaluated cumulative safety and other clinical trial data. Major adverse cardiac events and malignancies were assessed by an independent adjudication committee.

### Study Formulations

Benralizumab 20 mg/mL, 30 mg/mL, or 100 mg/mL solutions for injection in accessorized prefilled syringes corresponding to 10 mg, 30 mg, and 100 mg were administered at the study center by subcutaneous injection every 4 weeks for the first three doses and then every 8weeks thereafter, with the last dose of benralizumab administered at Week 48. Matching placebo solution for injection in accessorized prefilled syringes were administered at the study center by a subcutaneous injection in the same schedule as benralizumab. Each study patient received two syringes with a fill volume of 1 mL and 0.5 mL, respectively, to achieve blinding in a double dummy fashion.

### Clinical Assessments

COPD Assessment Test: The COPD Assessment Test (CAT) is an eight-item patient-reported outcome (PRO) tool developed to measure the impact of COPD on health status. The instrument uses semantic differential six-point response scales that are defined by contrasting adjectives to capture the impact of COPD. Content includes items related to cough, phlegm, chest tightness, breathlessness going up hills/stairs, activity limitation at home, confidence leaving home, sleep, and energy. A CAT total score is the sum of item responses. Scores range from 0-40, with higher scores indicative of greater COPD impact on health status. The CAT test was completed by patients at the following study center visits: Visit 4 (Week 0), Visit 6 (Week 4), Visit 7 (Week 8), Visit 9 (Week 16), Visit 11 (Week 24), Visit 13 (Week 32), Visit 15 (Week 40), Visit 17 (Week 48), and Visit 19 (Week 56).

St. George's Respiratory Questionnaire: St. George's Respiratory Questionnaire (SGRQ) is a 50-item PRO instrument developed to measure the health status of patients with airway obstruction diseases. The questionnaire is divided into two parts: part 1 consists of eight items pertaining to the severity of respiratory symptoms in the preceding 4 weeks; part 2 consists of 42 items related to the daily activity and psychosocial impacts of the individual's respiratory condition. The SGRQ yields a total score and three domain scores (symptoms, activity, and impacts). The total score indicates the impact of disease on overall health status. This total score is expressed as a percentage of overall impairment, in which 100 represents the worst possible health status and 0 indicates the best possible health status. Likewise, the domain scores range from 0-100, with higher scores indicative of greater impairment. Specific details on the scoring algorithms were provided by the developer in a user manual.² The SGRQ was completed by patients at the following study center visits: Visit 4 (Week 0), Visit 6 (Week 4), Visit 7 (Week 8), Visit 9 (Week 16), Visit 11 (Week 24), Visit 13 (Week 32), Visit 15 (Week 40), Visit 17 (Week 48), and Visit 19 (Week 56).

Baseline Transitional Dyspnea Index: The Baseline/Transitional Dyspnea Index (BDI/TDI) is an instrument developed to provide a multidimensional measure of dyspnea in relation to activities of daily living. The BDI provides a measure of dyspnea at a single state, the baseline, and the TDI evaluates changes in dyspnea from the baseline state. The instrument consists of three components: functional impairment, magnitude of task, and magnitude of effort rated along a five-grade scale for BDI and a seven-grade scale for TDI. The BDI was completed by patients at study center Visit 4 (Week 0). The TDI was completed by patients at the following study center visits: Visit 6 (Week 4), Visit 7 (Week 8), Visit 9 (Week 16), Visit 11 (Week 24), Visit 13 (Week 32), Visit 15 (Week 40), Visit 17 (Week 48), and Visit 19 (Week 56).

The Modified Medical Research Council Dyspnea Scale: The mMRC dyspnea scale uses a simple grading system to assess a patient's level of dyspnea that consists of five statements about perceived breathlessness. It is an interviewer-administered ordinal scale on which patients rate their dyspnea according to five grades of increasing severity. The mMRC dyspnea scale was completed at study center Visit 4 (Week 0).

Exacerbations of Chronic Pulmonary Disease Tool—Patient-Reported Outcome: The EXACT-PRO/E-RS COPD is a 14-item PRO instrument developed to assess the frequency, severity, and duration of COPD exacerbations. The instrument was developed for daily at-home administration via a handheld electronic device. Respondents are instructed to complete the diary at each evening just prior to bedtime and to answer the questions while considering their experiences “today.” The scores range between 0-100, with higher scores indicative of severity. Patients completed the EXACT-PRO/E-RS COPD at home every day in the evening.

EuroQOL 5D 5L: EuroQOL 5D 5L assesses five dimensions: mobility, self-care, usual activities, pain/discomfort, and anxiety/depression. Each dimension has five response options (no problems, slight problems, moderate problems, severe problems, and extreme problems) that reflect increasing levels of difficulty. Patients completed the EuroQOL 5D 5L at home on a weekly basis (±2 days).

### Safety Assessments

Baseline data for a complete physical examination were collected at Visit 1. Any new findings or aggravated existing findings were reported as an adverse event. In addition to physical examinations, the following were recorded at each visit: smoking status, anthropometric measures, vital signs. A 12-lead ECG was also performed in the supine position, after the participant had been resting for at least 5 minutes. The ECG was to be performed between interventions.

Laboratory safety assessments were also conducted with regards to clinical chemistry, hematology, and urinalysis. Clinical chemistry characteristics included the following: alkaline phosphatase, ALT, AST, blood urea nitrogen, calcium, chloride, carbon dioxide, creatinine, gamma glutamyl transpeptidase, glucose, phosphorus, sodium, total bilirubin, total cholesterol, and uric acid.

Hematology analyses included hematocrit, hemoglobin, mean corpuscular volume, platelet count, red blood cell count, and white blood cell count with differential.

Urinalysis included the following: appearance, blood, color, glucose, ketones, microscopy for white blood cells and red blood cells, pH, and specific gravity.

### Statistical Analysis

In the primary analysis, the number of COPD exacerbations observed for a patient during the 56-week double-blind treatment period was used as response variable.

$$Annual Exacerbation Rate = Number of Exacerbations * 365.25 / ( Last Follow ‐ up Date - Visit 4 Date + 1 )$$

Exacerbation rate in each of the benralizumab dosage groups was compared with exacerbation rate in the placebo group via a negative binomial model. The model included covariates of treatment group, eosinophil cohort (220-299/μL or ≥300/μL), country, background therapy (ICS/LABA, LABA/LAMA, or ICS/LABA/LAMA), and the number of exacerbations in the year before the study. The logarithm of the follow-up time was used as an offset variable in the model.

All secondary efficacy endpoints were analyzed for patients with baseline blood eosinophil counts ≥220/μL. Change from baseline in predose/prebronchodilator FEV₁ at Week 56 was compared between each of the three benralizumab dose groups and placebo via a repeated measures analysis on patients with a baseline predose/prebronchodilator FEV₁ and at least one post-randomization predose/prebronchodilator FEV₁ in the full analysis set. The dependent variable was the change from baseline in prebronchodilator FEV₁ at postbaseline protocol-specified visits (up to the end-of-treatment visit). Treatment group was fitted as the explanatory variable, and eosinophil cohort (220-299/μL or ≥300/μL), country, background therapy (ICS/LABA, LABA/LAMA, or ICS/LABA/LAMA), visit, the interaction between visit and treatment, and baseline prebronchodilator FEV₁ were fitted as covariates. Visit was fitted as a categorical variable, and the variance covariance matrix was assumed to be unstructured. The model was:

$$Change in {FEV}_{1} = Treatment Group + Eosinophil Cohort ( 220 - 299 / μL or \geq 300 / μL ) + Baseline {FEV}_{1} + Country + Background Therapy ( ICS / LABA , LABA / LAMA , or ICS / LABA / LAMA ) + Visit + Treatment * Visit$$

Change from baseline in SGRQ total score and three domain scores (symptoms, activity, and impacts) at Week 56 was analyzed separately via a similar model as the above model for change from baseline in predose/prebronchodilator FEV₁.

Sensitivity analyses for the primary endpoint and the key secondary endpoints based on different missing data mechanism assumptions, including those expected to be more conservative such as missing not at random, were used to explore the robustness of any treatment effect, including multiple imputation approaches.

To account for multiplicity to test the primary (exacerbation rate) and two key secondary endpoints (FEV₁ and SGRQ) for each of the 30 mg and 100 mg dosage groups (for patients with baseline blood eosinophil counts ≥220/μL), the following testing strategy was used to control the overall type I error rate at level 0.05:
Step 1: Two tests of annual COPD exacerbation rate were performed (one test for each dose vs. placebo) at the family-wise error rate (FWER) of 0.04 via a Hochberg procedure. If both p-values were <0.04, then Step 2 was undertaken; if the smaller p-value was <0.02, then Step 2a was performed. Otherwise, no null hypothesis was rejected.
Step 2: The two key secondary endpoints for both dosages were tested as one family at the FWER of 0.05 via a Holm procedure.
Step 2a: The two secondary endpoints for the smaller P-value dose were tested at the FWER of 0.01 via a Holm procedure.

### Secondary Endpoints

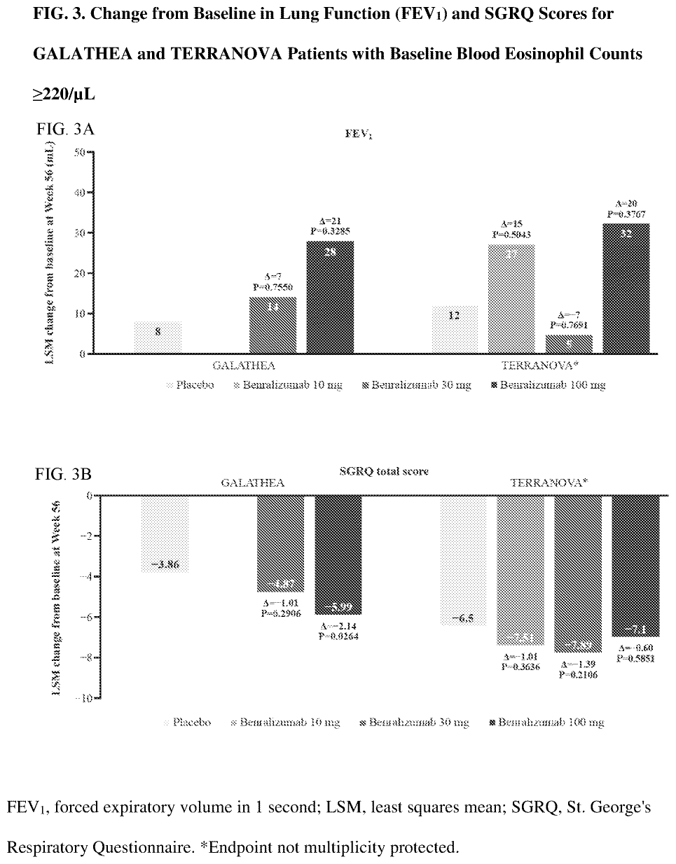

There were reported improvements in SGRQ scores for patients in both studies; however, an overall improvement in SGRQ scores was reported for patients from the placebo group as well as all treatment groups (least squares mean change from baseline, GALATHEA, placebo [−3.86] vs. 10 mg [0.00] vs. 30 mg [−4.87] vs. 100 mg [−5.99] and TERRANOVA, placebo [−6.50] vs. 10 mg [−7.51] vs. 30 mg [−7.89] vs. 100 mg [−7.10] (FIG. 3).

### Patients

GALATHEA and TERRANOVA included patients aged 40-85 years with 1) moderate to very severe COPD (screening post-bronchodilator FEV₁/forced vital capacity <0.70 and post-bronchodilator FEV₁>20% and ≤65% of predicted normal); 2) cumulative or past smoking exposure ≥10 pack-years; 3) symptoms (modified Medical Research Council dyspnea scale scores ≥1 at screening); and 4) documented history of ≥2 moderate COPD exacerbations requiring treatment with oral corticosteroids and/or antibiotics, or ≥1 COPD exacerbation requiring hospitalization within the year before enrollment despite treatment with double therapy (ICS/LABA or LABA/LAMA) or triple therapy (ICS/LABA/LAMA) throughout the previous year. Patients were excluded if they had a primary diagnosis of asthma (prior history of asthma [e.g., in childhood or adolescence] was permitted).

### Exclusion Criteria

Patients must not have entered the study if any of the following exclusion criteria were fulfilled:
Clinically important pulmonary disease other than COPD (e.g., active lung infection, clinically significant bronchiectasis, pulmonary fibrosis, cystic fibrosis, hypoventilation syndrome associated with obesity, lung cancer, alpha 1 anti-trypsin deficiency, and primary ciliary dyskinesia) or another diagnosed pulmonary or systemic disease that is associated with elevated peripheral eosinophil counts (e.g., allergic bronchopulmonary aspergillosis/mycosis, Churg-Strauss syndrome, hypereosinophilic syndrome) and/or radiological findings suggestive of a respiratory disease other than COPD that is contributing to the respiratory symptoms of the patient;
Any disorder, including, but not limited to, cardiovascular, gastrointestinal, hepatic, renal, neurological, musculoskeletal, infectious, endocrine, metabolic, hematological, psychiatric, or major physical impairment that was not stable in the opinion of the investigator and/or could:
   Affect the safety of the patient throughout the study;
   Influence the findings of the study or their interpretation; or
   Impede the patient's ability to complete the duration of study.
Treatment with systemic corticosteroids and/or antibiotics, and/or hospitalization for a COPD exacerbation within 2 weeks prior to enrollment (Visit 1) or during the enrollment and screening/run-in period, based on last dose of steroids or last date of hospitalization, whatever occurred later.
Acute upper or lower respiratory infection within 2 weeks prior to enrollment (Visit 1) or during the enrollment and screening/run-in period.
Pneumonia within 8 weeks prior to enrollment (Visit 1), based on the last day of antibiotic treatment or hospitalization date, whatever occurred later or during the enrollment and screening/run-in period.

### Statistical Analysis

For sample size calculations, an estimated 348 patients with baseline blood eosinophil counts ≥220/μL per treatment arm (overall total patients: GALATHEA, 1,044; TERRANOVA, 1,392) were required for the primary endpoint to provide 90% power of detecting a 30% reduction between annual exacerbation rate in the benralizumab 30 mg or 100 mg groups versus placebo with assumption of two-sided 4% alpha level tests.

Exacerbation rates were compared for benralizumab groups versus placebo with a negative binomial model for patients in the primary analysis population with baseline blood eosinophil counts ≥220/μL. The model response variable was number of exacerbations over the 56-week treatment period. The model included covariates of treatment group, eosinophil count cohort (220-299/μL or >300/μL), region, background therapy (ICS/LABA, LABA/LAMA, ICS/LABA/LAMA), and number of exacerbations in the previous year. Multiplicity protection of the overall type I error rate was accounted for through the Hochberg procedure. In TERRANOVA, the benralizumab 10 mg treatment arm was not multiplicity protected.

Secondary efficacy end points were analyzed for patients with baseline blood eosinophil counts ≥220/μL. Analysis of secondary end points was not multiplicity protected. Change from baseline in prebronchodilator FEV₁ at Week 56 was compared between each of the benralizumab dosage groups and placebo via a repeated measures analysis. Change from baseline in SGRQ total score at Week 56 was analyzed separately with a similar model. Safety parameters were analyzed by means of count summaries by study period.

Prespecified subgroup analyses by patient demographics, respiratory disease characteristics, prior medication use, and prior exacerbations (<3 or ≥3) were performed for the primary efficacy variable for each trial. The study was not designed or powered to assess efficacy within these subgroups. These analyses are therefore considered exploratory.

### Example 2—Results

### Study Population

Overall, GALATHEA and TERRANOVA patient characteristics and demographics were similar to those of the primary analysis population with baseline blood eosinophil counts ≥220/μL (Table 1). Most patients were white men, and the average age was 65 years. The rates of current (nonprimary diagnosis) and past asthma were low (<7% and ≤10%, respectively, in all study groups) (Table 1). Most patients (93.0%) were classified as Global Initiative for COPD (GOLD) group D at baseline. The percentage of patients who changed background maintenance therapy during the on-treatment or post-treatment period was low (<5% and <1%, respectively). All results reported are for the primary analysis population (GALATHEA, n=1,120; TERRANOVA, n=1,545).

In GALATHEA and TERRANOVA, ≥83% and ≥79% of patients, respectively, in all treatment arms completed treatment. Primary reasons for treatment discontinuation were AEs and patient decision (data not shown).

TABLE 1
Demographics and Baseline Clinical Characteristics of GALATHEA and
TERRANOVA Patients with COPD and Baseline Blood Eosinophil Counts ≥220/μL

| | GALATHEA | | | TERRANOVA | | | |
|---|---|---|---|---|---|---|---|
| | Benralizumab | Benralizumab | | Benralizumab | Benralizumab | Benralizumab | |
| | 30 mg Q8W | 100 mg Q8W | Placebo | 10 mg Q8W | 30 mg Q8W | 100 mg Q8W | Placebo |
| | n = 382 | n = 379 | n = 359 | n = 377 | n = 394 | n = 386 | n = 388 |
| Age, years, mean (SD) | 65.8 (7.95) | 65.5 (8.29) | 65.6 (8.56) | 65.1 (8.49) | 65.9 (8.26) | 64.9 (8.17) | 65.0 (8.38) |
| Male sex, n (%) | 270 (70.7) | 262 (69.1) | 260 (72.4) | 252 (66.8) | 269 (68.3) | 250 (64.8) | 253 (65.2) |
| Race, n (%) | | | | | | | |
| White | 339 (88.7) | 334 (88.1) | 315 (87.7) | 300 (79.6) | 312 (79.2) | 296 (76.7) | 303 (78.1) |
| Black or African-American | 2 (0.5) | 9 (2.4) | 7 (1.9) | 6 (1.6) | 7 (1.8) | 13 (3.4) | 14 (3.6) |
| Asian | 36 (9.4) | 35 (9.2) | 33 (9.2) | 46 (12.2) | 49 (12.4) | 55 (14.2) | 50 (12.9) |
| Other | 5 (1.3) | 1 (0.3) | 4 (1.1) | 25 (6.6) | 26 (6.6) | 22 (5.7) | 21 (5.4) |
| BMI, kg/m², mean (SD) | 27.37 (5.672) | 27.81 (6.174) | 27.37 (6.069) | 26.81 (5.566) | 27.15 (6.668) | 26.82 (5.804) | 26.51 (5.737) |
| Baseline eosinophil count, | 451.3 (281.5) | 458.5 (276.6) | 449.8 (283.6) | 518.2 (420.12) | 503.2 (388.55) | 504.2 (403.63) | 492.9 (359.55) |
| cells/μL, mean (SD) | | | | | | | |
| Maintenance therapy, n (%) | | | | | | | |
| ICS/LABA | 72 (18.8) | 80 (21.1) | 86 (24.0) | 131 (34.7) | 127 (32.2) | 133 (34.5) | 133 (34.3) |
| LABA/LAMA | 33 (8.6) | 37 (9.8) | 31 (8.6) | 29 (7.7) | 42 (10.7) | 16 (4.1) | 25 (6.4) |
| ICS/LABA/LAMA | 276 (72.3) | 262 (69.1) | 242 (67.4) | 216 (57.3) | 224 (56.9) | 237 (61.4) | 229 (59.0) |
| Number of patients | 2.31 (1.17) | 2.33 (1.15) | 2.35 (1.42) | 2.3 (1.02) | 2.2 (0.98) | 2.3 (1.01) | 2.3 (1.0) |
| with exacerbations in | | | | | | | |
| the previous 12 | | | | | | | |
| months, mean (SD) | | | | | | | |
| 1,^{a} n (%) | 70 (18.3) | 62 (16.4) | 69 (19.2) | 48 (12.7) | 62 (15.7) | 50 (13.0) | 45 (11.6) |
| 2 | 205 (53.7) | 210 (55.4) | 198 (55.2) | 244 (64.7) | 234 (59.4) | 235 (60.9) | 232 (59.8) |
| 3 | 65 (17.0) | 61 (16.1) | 52 (14.5) | 54 (14.3) | 71 (18.0) | 62 (16.1) | 75 (19.3) |
| 4 | 24 (6.3) | 29 (7.7) | 17 (4.7) | 17 (4.5) | 15 (3.8) | 24 (6.2) | 21 (5.4) |
| 5 | 5 (1.3) | 8 (2.1) | 9 (2.5) | 9 (2.4) | 5 (1.3) | 7 (1.8) | 9 (2.3) |
| 6 | 7 (1.8) | 5 (1.3) | 3 (0.8) | 0 | 4 (1.0) | 6 (1.6) | 2 (0.5) |
| 7 | 4 (1.0) | 2 (0.5) | 5 (1.4) | 3 (0.3) | 1 (0.3) | 1 (0.3) | 1 (0.3) |
| ≥8 | 2 (0.5) | 2 (0.5) | 6 (1.7) | 2 (0.5) | 2 (0.5) | 1 (0.3) | 3 (0.8) |
| ≥1 exacerbation in | 152 (39.8) | 153 (40.4) | 147 (40.9) | 112 (29.7) | 142 (36.0) | 140 (36.3) | 130 (33.5) |
| the prior year resulting | | | | | | | |
| in hospitalization, n (%) | | | | | | | |
| Pre-BD FEV₁, mean % | 39.7 (13.1) | 40.5 (12.5) | 41.1 (14.9) | 42.7 (14.3) | 40.5 (13.2) | 40.9 (13.6) | 41.2 (13.0) |
| predicted normal (SD) | | | | | | | |
| Post-BD FEV₁, mean % | 42.4 (11.8) | 43.5 (12.0) | 43.4 (12.8) | 43.7 (11.8) | 42.6 (12.1) | 42.5 (12.3) | 42.9 (11.5) |
| predicted normal (SD) | | | | | | | |
| Post-BD FEV₁/ | 43 (11) | 44 (11) | 44 (12) | 45 (11) | 44 (11) | 45 (11) | 45 (11) |
| FVC, mean % (SD) | | | | | | | |
| Post-BD FEV₁, L, mean (SD) | 1.203 (0.416) | 1.234 (0.423) | 1.237 (0.448) | 1.204 (0.406) | 1.152 (0.383) | 1.175 (0.426) | 1.171 (0.393) |
| Diagnosis of obstructive | 301 (78.8) | 283 (74.7) | 265 (73.8) | 242 (64.2) | 251 (63.7) | 263 (68.1) | 257 (66.2) |
| chronic bronchitis,^{b} n (%) | | | | | | | |
| Diagnosis of chronic | 211 (55.2) | 211 (55.7) | 206 (57.4) | 196 (52.0) | 224 (56.9) | 215 (55.7) | 189 (48.7) |
| emphysema,^{c} n (%) | | | | | | | |
| Current diagnosis | 17 (4.5) | 26 (6.9) | 18 (5.0) | 6 (1.6) | 17 (4.3) | 12 (3.1) | 16 (4.1) |
| of asthma, n (%) | | | | | | | |
| Previous diagnosis | 26 (6.8) | 38 (10.0) | 29 (8.1) | 18 (4.8) | 21 (5.3) | 28 (7.3) | 28 (7.2) |
| of asthma, n (%) | | | | | | | |

^{a}Patients could enter the studies with one prior exacerbation only if it was severe (resulted in hospitalization).
^{b}Cough and mucus production for at least 3 months in 2 successive years.
^{c}Historical or recent evidence of emphysema by chest x-ray or computerized tomography scan.
AE, adverse event; BMI, body mass index; COPD, chronic obstructive pulmonary disease; FEV₁, forced expiratory volume in 1 second; FVC, forced vital capacity; ICS, inhaled corticosteroids; LABA, long-acting β₂-agonists; LAMA, long-acting muscarinic antagonists; pre-BD, prebronchodilator; post-BD, postbronchodilator; Q8W, every 8 weeks (first three doses every 4 weeks); SD, standard deviation.

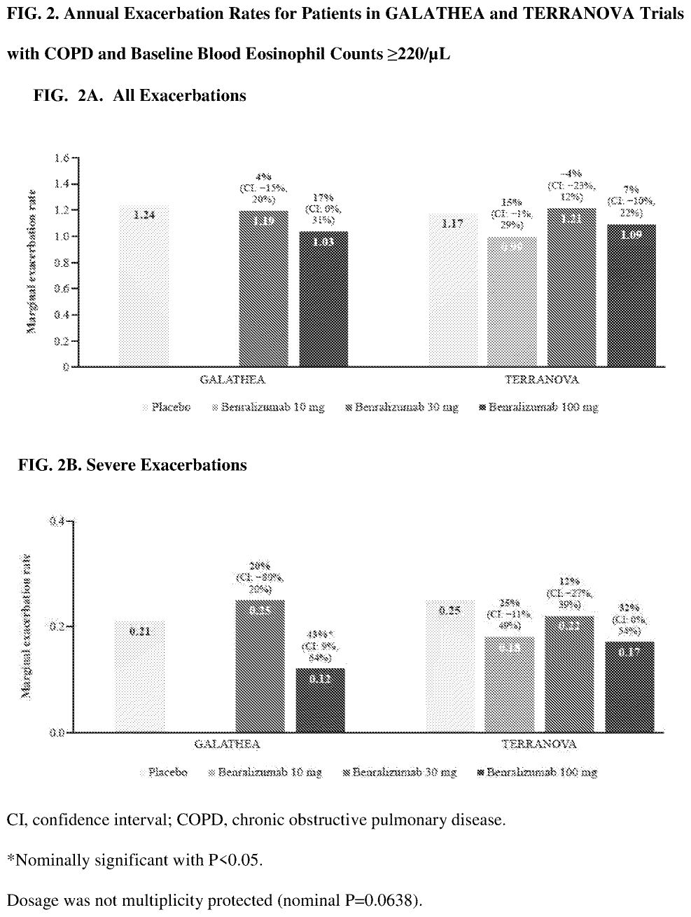

Primary End Points: In GALATHEA, patients treated with benralizumab had a reduction in annual COPD exacerbation rate at Week 56 compared with placebo. Annualized exacerbation rate estimates (95% confidence intervals [CI]) were 1.19 (1.04, 1.36), 1.03 (0.90, 1.19), and 1.24 (1.08, 1.42) for benralizumab 30 mg, 100 mg, and placebo, respectively: treatment effect versus placebo was 0.96 (P=0.6490) and 0.83 (P=0.0525) for benralizumab 30 mg and 100 mg, respectively (Table 2, FIG. 2). Benralizumab 100 mg also reduced annual rate of severe exacerbations (treatment effect vs. placebo, 0.57 [nominal P=0.0175]) (Table 2, FIG. 2).

In TERRANOVA, patients treated with benralizumab 10 mg or 100 mg had a reduction in annual COPD exacerbation rate at Week 56 compared with placebo. Annualized exacerbation rate estimates (95% CI) were 0.99 (0.87, 1.13), 1.21 (1.08, 1.37), 1.09 (0.96, 1.23), and 1.17 (1.04, 1.32) for benralizumab 10 mg, 30 mg. 100 mg, and placebo, respectively: treatment effect versus placebo was 0.85 (P=0.0638), 1.04 (P=0.6575), and 0.93 (P=0.3988) for benralizumab 10 mg, 30 mg, and 100 mg, respectively (Table 2). Numerical differences favoring benralizumab were also observed for reduction in annual rate of severe exacerbations (treatment effect vs. placebo, 0.75 [nominal P=0.1478], 0.88 [nominal P=0.4835], and 0.68 [nominal P=0.0516] for benralizumab 10 mg. 30 mg, and 100 mg, respectively) (Table 2, FIG. 2).

Key Secondary Outcomes: No secondary outcome differences were statistically significant at Week 56 for benralizumab compared with placebo. In GALATHEA, change from baseline in pre-bronchodilator FEV₁ versus placebo was 7 mL and 21 mL for benralizumab 30 mg and 100 mg, respectively. For SGRQ total score, benralizumab treatment effect versus placebo was −1.011 and −2.136 for benralizumab 30 mg and 100 mg, respectively (Table 2,and FIG. 3). In TERRANOVA, changes from baseline in pre-bronchodilator FEV₁ versus placebo were 15 mL, −7 mL, and 20 mL for benralizumab 10 mg, 30 mg, and 100 mg, respectively. For SGRQ total score, benralizumab treatment effect versus placebo was −1.011, −1.388, and −0.602 for benralizumab 10 mg, 30 mg, and 100 mg, respectively (Table 2, and FIG. 3). At Week 56, there were also large improvements in SGRQ scores for patients randomized to placebo in GALATHEA and TERRANOVA (mean change from baseline −3.856 and −6.863, respectively).

TABLE 2
Efficacy of Benralizumab in the Phase III GALATHEA and TERRANOVA
Trials for Patients with COPD and Baseline Blood Eosinophil Counts ≥220/μL

| | | GALATHEA | | | TERRANOVA | | | |
|---|---|---|---|---|---|---|---|---|
| | | Benralizumab | Benralizumab | | Benralizumab | Benralizumab | Benralizumab | |
| | | 30 mg Q8W | 100 mg Q8W | Placebo | 10 mg Q8W | 30 mg Q8W | 100 mg Q8W | Placebo |
| Annual exacerbation rate | N | 382 | 379 | 359 | 377 | 394 | 386 | 388 |
| ratio, benralizumab vs. | Treatment effect | 0.96 | 0.83 | | 0.85 | 1.04 | 0.93 | |
| placebo^{a} | (95% CI) | (0.80, 1.15) | (0.69, 1.00) | | (0.71, 1.01) | (0.88, 1.23) | (0.78, 1.10) | |
| | P-value | 0.6490 | 0.0525 | | 0.0638 | 0.6575 | 0.3988 | |
| Annual severe | N | 382 | 379 | 359 | 377 | 394 | 386 | 388 |
| exacerbation rate ratio, | Treatment effect | 1.20 | 0.57 | | 0.75 | 0.88 | 0.68 | |
| benralizumab vs. placebo^{b} | (95% CI) | (0.80, 1.80) | (0.36, 0.91) | | (0.51, 1.11) | (0.61, 1.27) | (0.46, 1.00) | |
| | P-value | 0.3861 | 0.0175 | | 0.1478 | 0.4835 | 0.0516 | |
| Pre-BD FEV₁(L) change | N | 381 | 376 | 356 | 325 | 322 | 347 | 344 |
| from baseline, LS mean | Treatment effect | 0.007 | 0.021 | | 0.015 | −0.007 | 0.020 | |
| difference, benralizumab | (95% CI) | (−0.035, 0.048) | (−0.021, 0.062) | | (−0.029, 0.059) | (−0.051, 0.037) | (−0.024, 0.064) | |
| vs. placebo^{c} | P-value | 0.7550 | 0.3285 | | 0.5043 | 0.7691 | 0.3767 | |
| SGRQ total change from | N | 381 | 373 | 355 | 331 | 329 | 354 | 349 |
| baseline, LS mean | Treatment effect | −1.011 | −2.136 | | −1.011 | −1.388 | −0.602 | |
| difference, benralizumab | (95% CI) | (−2.887, 0.865) | (−4.020, −0.251) | | (−3.192, 1.171) | (−3.562, 0.786) | (−2.763, 1.560) | |
| vs. placebo^{c} | P-value | 0.2906 | 0.0264 | | 0.3636 | 0.2106 | 0.5851 | |

CI, confidence interval; COPD, chronic obstructive pulmonary disease; FEV₁, forced expiratory volume in 1 second; LS, least squares; pre-BD, prebronchodilator; Q8W, every 8 weeks (first three doses every 4 weeks); SGRQ, St. George's Respiratory Questionnaire (50-item health-related quality of life questionnaire; score range: 100 = worse, 0 = best).
^{a}Negative binomial model adjusting for treatment, eosinophil count cohort (220-299/μL, ≥300/μL), geographic region, background therapy (inhaled corticosteroids [ICS]/long-acting β₂-agonists [LABA], LABA/long-acting muscarinic antagonists [LAMA], ICS/LABA/LAMA), and number of prior exacerbations.
^{b}Not adjusted for multiplicity, nominal P-value.
^{c}Gatekeeping procedure strategy to control for overall type I error at level 0.05; mixed-effects model for repeated measures adjusting for treatment, baseline value, eosinophil count cohort (220-299/μL, ≥300/μL), geographic region, and background therapy (ICS/LABA, LABA/LAMA, ICS/LABA/LAMA).

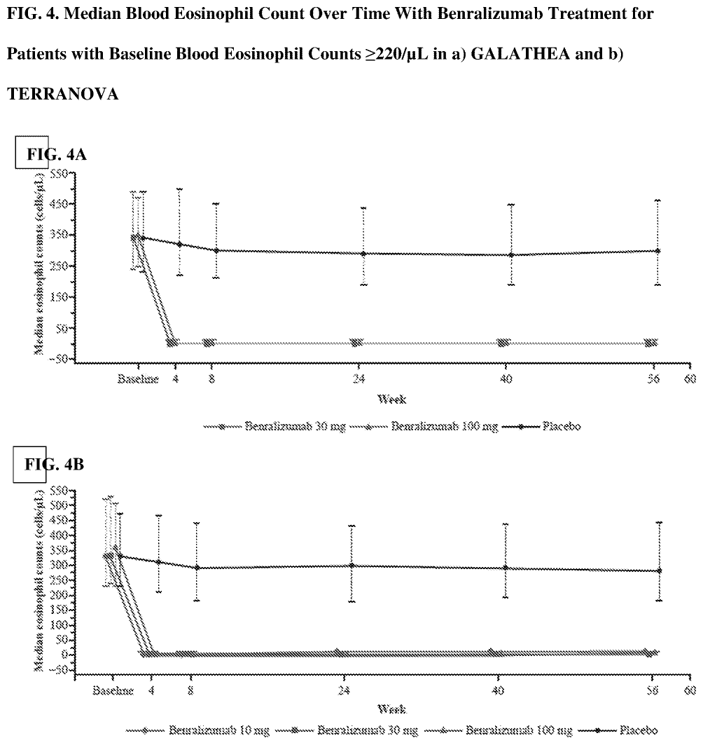

In both trials, all benralizumab dosages led to complete depletion of blood eosinophils from Week 4 to study end versus no depletion with placebo (FIG. 4). In a GALATHEA substudy, benralizumab produced nearly complete depletion at Week 24 (mean: 0.02% and 0.13% for 30 mg and 100 mg, respectively, vs. 5.38% for placebo), maintained to Week 56.

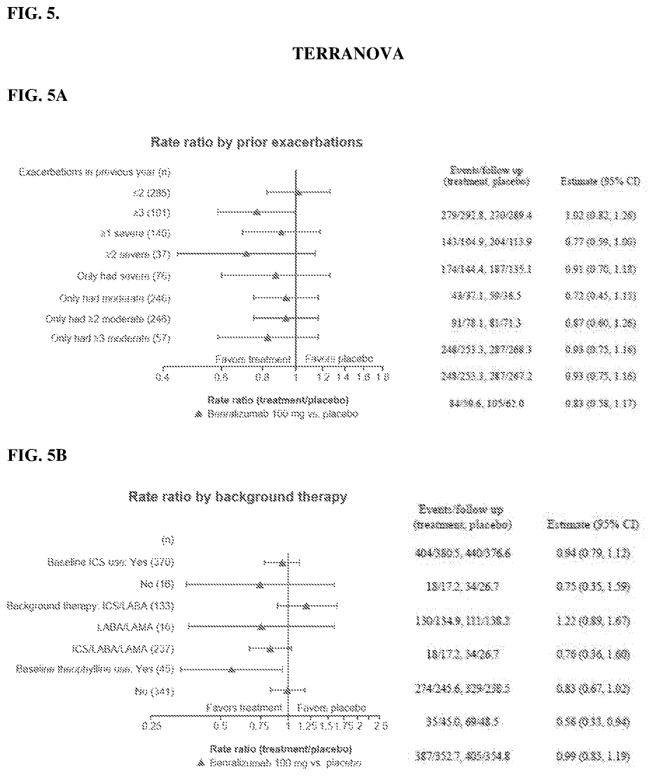

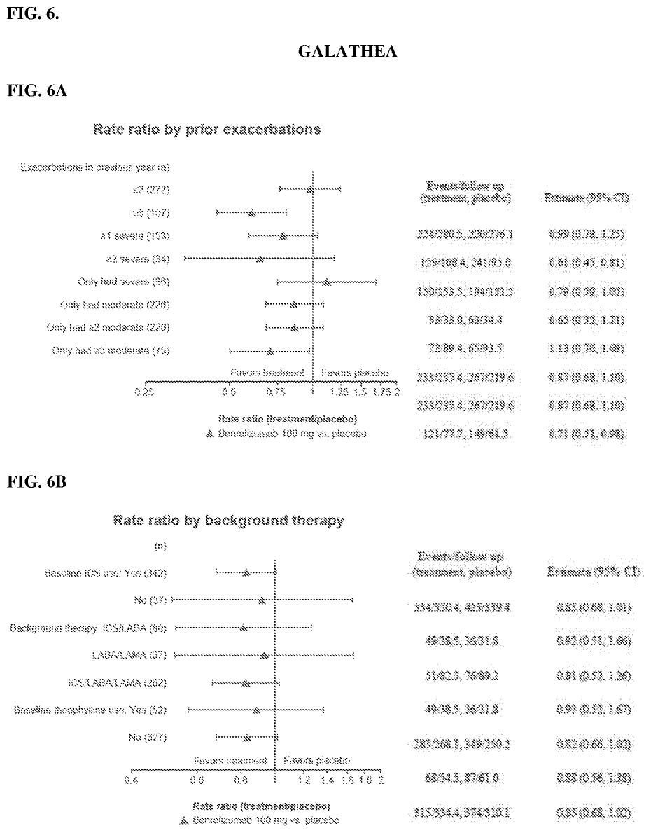

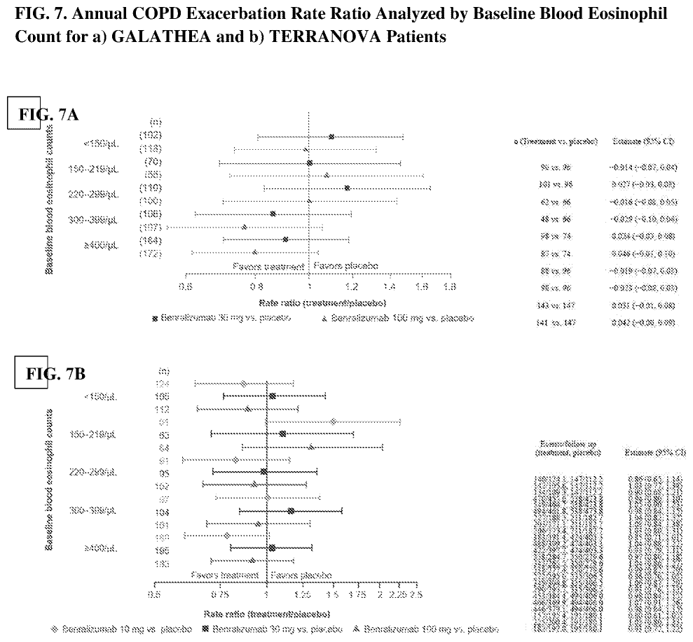

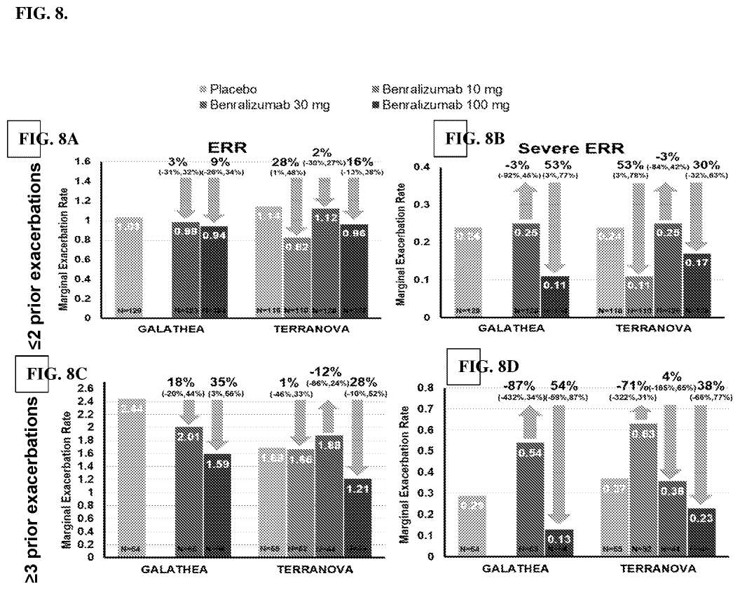

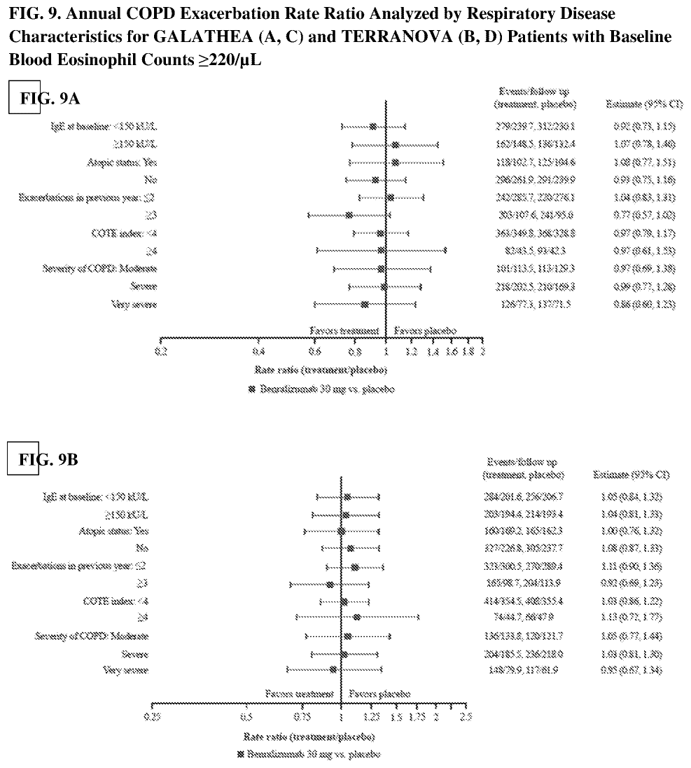

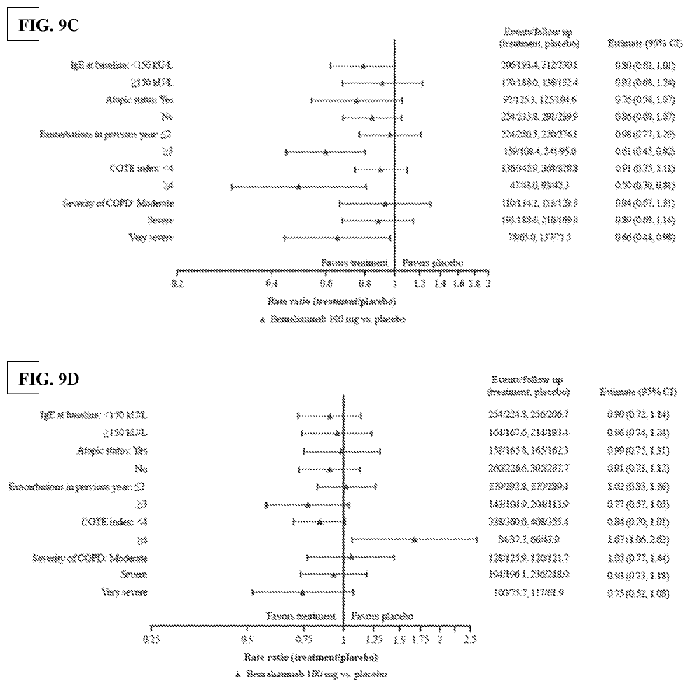

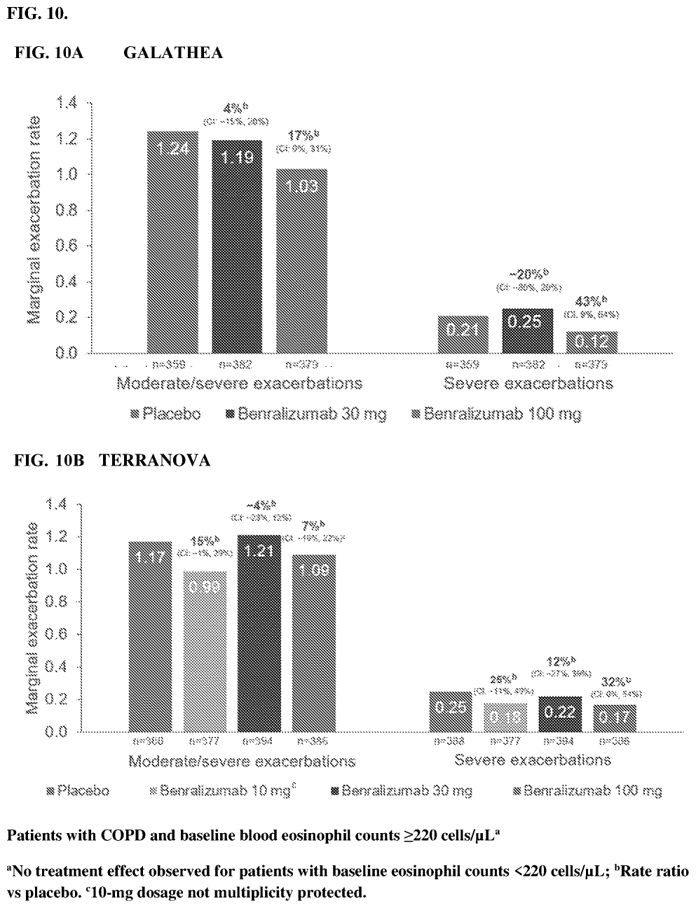

Pre-specified Subgroup Analyses: Point estimates for the annual COPD exacerbation rate ratio (RR) favored benralizumab 100 mg over placebo for all demographic subgroups, except for current smokers in GALATHEA, who had similar results with benralizumab and placebo (point estimate of 1.00). Subgroups of patients with ≥3 compared with <3 prior exacerbations had a greater treatment effect with benralizumab 100 mg (reduction vs. placebo: GALATHEA, 39% and 2%, respectively: TERRANOVA, 23% and 2%, respectively) (FIGS. 5 and 6). Greater exacerbation rate reductions with benralizumab 100 mg were demonstrated for patients with baseline eosinophil counts ≥220/μL (RR vs. placebo [95% CI]: GALATHEA, 0.83 [0.69, 1.00]: TERRANOVA, 0.93 [0.78, 1.10]) compared with <220/μL (RR vs. placebo [95% CI]: GALATHEA, 1.02 [0.82, 1.27]: TERRANOVA, 1.02 [0.80, 1.30]). There was no trend between increasing eosinophil counts and treatment effects (FIG. 7).

Benralizumab 100 mg produced an increased treatment effect for patients who had been receiving triple therapy compared with other treatment regimens (COPD exacerbation rate reduction vs. placebo: GALATHEA, 18%: TERRANOVA, 17%) (FIGS. 5 and 6). In both trials, patients who had frequent prior exacerbations (≥3) or very severe GOLD airflow severity responded with greater benralizumab treatment effect versus patients with few prior exacerbations or moderate GOLD airflow severity. Benralizumab treatment effect varied marginally by region (data not shown).

Safety: In GALATHEA and TERRANOVA, AE types and frequencies were similar between benralizumab and placebo groups and death rates were low (Table 3). The most common AEs were related to COPD or respiratory conditions. Fewer than 15% of patients had a positive anti-drug antibody (ADA) response (Table 3), and this appeared to have no impact on clinical outcomes.

TABLE 3
Safety of Benralizumab in the Phase III GALATHEA and TERRANOVA
Trials for Patients with COPD and Baseline Blood Eosinophil Counts ≥220/μL

| | GALATHEA | | | TERRANOVA | | | |
|---|---|---|---|---|---|---|---|
| | Benralizumab | Benralizumab | | Benralizumab | Benralizumab | Benralizumab | |
| Preferred term, n (%) | 30 mg Q8W | 100 mg Q8W | Placebo | 10 mg Q8W | 30 mg Q8W | 100 mg Q8W | Placebo |
| Patients with any AE | 427 (77.1) | 445 (80.6) | 421 (76.5) | 395 (70.4) | 424 (75.3) | 397 (70.6) | 406 (71.5) |
| AE leading to death | 15 (2.7) | 11 (2.0) | 13 (2.4) | 17 (3.0) | 21 (3.7) | 17 (3.0) | 19 (3.3) |
| Any SAE | 151 (27.3) | 177 (32.1) | 176 (32.0) | 144 (25.7) | 177 (31.4) | 127 (22.6) | 158 (27.8) |
| AE leading to | 30 (5.4) | 33 (6.0) | 26 (4.7) | 20 (3.6) | 33 (5.9) | 26 (4.6) | 16 (2.8) |
| discontinuation | | | | | | | |
| COPD | 98 (17.7) | 83 (15.0) | 105 (19.1) | 97 (17.3) | 113 (20.1) | 85 (15.1) | 93 (16.4) |
| Viral upper respiratory | 83 (15.0) | 95 (17.2) | 66 (12.0) | 62 (11.1) | 47 (8.3) | 60 (10.7) | 70 (12.3) |
| tract infection | | | | | | | |
| Bronchitis | 60 (10.8) | 86 (15.6) | 83 (15.1) | 66 (11.8) | 73 (13.0) | 64 (11.4) | 66 (11.6) |
| Upper respiratory | 69 (12.5) | 75 (13.6) | 66 (12.0) | 68 (12.1) | 71 (12.6) | 68 (12.1) | 67 (11.8) |
| tract infection | | | | | | | |
| Lower respiratory | 50 (9.0) | 32 (5.8) | 29 (5.3) | 26 (4.6) | 23 (4.1) | 15 (2.7) | 21 (3.7) |
| tract infection | | | | | | | |
| Pneumonia | 32 (5.8) | 29 (5.3) | 24 (4.4) | 28 (5.0) | 33 (5.9) | 22 (3.9) | 38 (6.7) |
| Urinary tract infection | 24 (4.3) | 23 (4.2) | 15 (2.7) | 25 (4.5) | 26 (4.6) | 28 (5.0) | 19 (3.3) |
| ADA prevalence | 40 (10.5) | 48 (12.7) | 27 (7.5) | 52 (13.8) | 41 (10.4) | 59 (15.3) | 22 (5.7) |

ADA, anti-drug antibody; AE; adverse event; COPD, chronic obstructive pulmonary disease; Q8W, every 8 weeks (first three doses every 4 weeks); SAE, serious adverse event.

### Discussion:

Although not statistically significant compared with placebo, benralizumab as an add-on maintenance treatment for patients with moderate to very severe COPD receiving ICS/LABA, LABA/LAMA, or ICS/LABA/LAMA inhaled therapy with a history of COPD exacerbations and blood eosinophil counts ≥220/μL demonstrated evidence of reducing exacerbation rates. Subgroup analyses in both studies consistently identified patients with a blood eosinophil count of ≥300 eosinophils/μL, with a history of more frequent exacerbations (e.g., ≥3 exacerbations in the year prior to administration), and those receiving triple background therapy (e.g., ICS/LABA/LAMA) as most likely to respond to benralizumab 100 mg treatment. Furthermore, annual rate of severe COPD exacerbations was reduced by >30% with benralizumab 100 mg compared with placebo.

Most benralizumab dosages were associated with a small improvement from baseline in lung function. Benralizumab treatment also improved SGRQ score, as did placebo. All patients received on-study inhaled therapy that could have differentially improved placebo group outcomes. This, in combination with the large placebo effect, may potentially have contributed to benralizumab failing to reach statistical significance for improvements in lung function and other end points.

During these trials, AEs/serious AEs were balanced across treatment arms and deaths rates were low. These safety data are consistent with those reported in the Phase III trials of benralizumab for severe, uncontrolled eosinophilic asthma (Bleecker et al., Lancet, 388:2115-2127, 2016; Fitzgerald et al., Lancet, 388:2128-2142, 2016).

COPD is a complex condition driven by a diverse range of mechanisms, which leads to a wide spectrum of clinical disease presentations (Singh et al., BRN Review, 4:34-52, 2018). Biologics such as benralizumab target a discrete potential pathological mechanism (eosinophilic inflammation). By Week 4, and through to Week 56, benralizumab resulted in complete blood and sputum eosinophil depletion. This finding, together with a similar effect on eosinophils observed in the mepolizumab trials, with minimal impact on COPD exacerbation rate, suggests that eosinophil depletion may not totally ameliorate exacerbation outcomes for patients with COPD. Furthermore, peripheral blood eosinophil counts may not accurately reflect airway eosinophilia. Clinical features of COPD are additional key factors. Both GALATHEA and TERRANOVA indicated that patients with more frequent COPD exacerbations in the year before enrollment despite receiving triple inhaled therapy experienced the greatest benralizumab treatment effect. We believe this to be an important finding because both variables are easily identified, and together with blood eosinophil counts could help personalize the use of benralizumab for the treatment of patients with COPD.

### Example 3—A Further Phase 3 Study

This is a randomized, double-blind, placebo-controlled, parallel-group, multicenter, Phase 3 study to evaluate the efficacy and safety of a benralizumab 100 mg dose administered by subcutaneous (SC) injection every 4 weeks for the first 3 doses and then every 8 weeks thereafter (hereafter referred to as Q8W) in patients with moderate to very severe COPD with a history of frequent COPD exacerbations and elevated peripheral blood eosinophils (≥300/μL). Eligible patients must have a history of ≥2 moderate and/or severe COPD exacerbations in the previous year despite receiving triple (ICS/LABA/LAMA) background therapy. Eligible patients must also have an elevated blood eosinophil count of ≥300/μL at screening supported by at least 1 historical result of ≥150/μL within the previous year.

In the previous Phase 3 studies, GALATHEA and TERRANOVA, long-term treatment with benralizumab 10, 30, or 100 mg Q8W through Week 48 was well tolerated in patients with moderate to very severe COPD. There were no appreciable differences in the safety results across the 3 dose groups.

The results from both studies provided consistent and clear evidence of a greater treatment effect for the benralizumab 100 mg dose over the 30 mg dose in reducing the annual rate of COPD exacerbations (moderate and severe combined) and severe exacerbations, as well as all other exacerbation endopoints, in patients with a baseline blood eosinophil count ≥220/μL. The benralizumab 10 mg dose (TERRANOVA only) provided inconsistent results compared with the 100 mg dose across exacerbation endpoints. However, for severe exacerbations, a greater treatment effect was observed for the benralizumab 100 mg dose compared with the 10 mg dose (32% and 25% reduction over placebo, respectively) in patients with a baseline blood eosinophil count ≥220/μL. In patients with more frequent prior exacerbations (≥3 exacerbations in the previous year) and a baseline blood eosinophil count ≥220/μL, a much greater treatment effect for the benralizumab 100 mg dose over the 10 mg dose was apparent for COPD exacerbations (moderate and severe combined: 23% vs 9% reduction over placebo, respectively) as well as for severe exacerbations (34% vs 17% reduction over placebo). In general, any improvements observed for benralizumab treated patients were greater for the 100 mg groups compared with the 30 mg groups. In both studies, benralizumab 100 mg reduced the annual rate of severe exacerbations by >30% compared to placebo. However, in GALATHEA, patients receiving benralizumab 30 mg had a greater severe exacerbation rate compared to placebo. These results suggest that a greater dosage of benralizumab may be needed to achieve treatment response for patients with COPD than patients with asthma, where benralizumab 30 mg is sufficient to achieve treatment effect in asthma. Considering the more consistent findings of efficacy, particularly in the subpopulation with more frequent exacerbations and elevated eosinophils (≥220/μL), the benralizumab 100 mg dose was chosen for this study.

Potentially eligible patients will enter the run-in period of 5 weeks. Patients who meet eligibility criteria will be randomized in a 1:1 ratio to receive either benralizumab 100 mg or placebo Q8W. The treatment period will be of variable duration and will continue until the last patient has the opportunity to complete a minimum of 56 weeks, at which point all patients will complete the study. The primary endpoint will be analyzed at Week 56.

At randomization, patients will be stratified by country and number of exacerbations in the previous year (2 or ≥3). Randomization to the stratum of 2 exacerbations in the previous year will be capped to ensure ≥70% of patients with ≥3 exacerbations in the previous year in the study population.

Patients will be maintained on triple (ICS/LABA/LAMA) background therapy from enrollment throughout the run-in and treatment period.

### Number of Patients:

A total of 868 patients are expected to be randomized in the study at a 1:1 ratio to either benralizumab 100 mg SC or matching placebo treatment groups.

### Treatments and Treatment Duration:

Once enrolled, patients will enter a run-in period with a minimum duration of 5 weeks that can be extended up to 13 weeks. Following the run-in period, patients confirmed to be eligible will be randomized in a 1:1 ratio to receive benralizumab 100 mg or placebo SC Q8W. The treatment period will be of variable duration and will continue until the last patient has the opportunity to complete a minimum of 56 weeks. When the end of treatment (EOT) visit has been identified for the last patient, then the final visits for all patients will be scheduled. The study will end when all patients complete their final visit.

### Statistical Methods

Efficacy analyses will be performed using the full analysis set (FAS), which will include all patients randomized who receive at least 1 dose of IP, irrespective of their protocol adherence and continued participation in the study, according to the Intent-to-Treat principle.

The primary efficacy endpoint is the annualized rate of moderate and severe COPD exacerbations over the first 56 weeks. A treatment policy estimand will be applied to the primary analysis of the primary endpoint (along with the main analysis of key secondary endpoints) whereby all data is included, regardless of whether a patient remains on blinded IP or not.

The primary endpoint will be assessed first in the primary population (patients with ≥3 exacerbations in the previous year) and then assessed on the overall population of patients with ≥2 exacerbations in the previous year. Exacerbation rate in the benralizumab 100 mg group will be compared to the exacerbation rate in the placebo group using a negative binomial model. The response variable in the model will be the number of COPD exacerbations over the first 56 weeks of the treatment period. The model will include covariates of treatment group, region, and number of exacerbations in the previous year. Thea logarithm of the follow-up time during the first 56 weeks will be used as an offset variable in the model. Hypothesis testing for the primary analysis will be performed at the 2-sided 5% significance level.

Annualized rate of COPD exacerbations leading to hospitalization or death (i.e. severe exacerbations) will be analyzed through EOT using a similar negative binomial model as outlined for the primary efficacy variable.

Unless otherwise noted, all secondary efficacy endpoints will be analyzed in patients with a history of ≥3 exacerbations in the previous year and repeated in patients with a history of ≥2 exacerbations in the previous year using data up to Week 56. Where serially measured endpoints (e.g. SGRQ and FEV1) are also collected beyond Week 56, additional models similar to those fitted on the data up to Week 56 will be used to assess these endpoints through EOT.

The study will recruit patients on triple (ICS/LABA/LAMA) background therapy with baseline eosinophil counts ≥300 eosinophils/μL and ≥2 exacerbations in the previous year, with at least 70% of patients with ≥3 exacerbations in the previous year. This study is powered for the primary population of patients with ≥3 exacerbations in the previous year. For the primary endpoint of annualized exacerbation rate over the first 56 weeks, a minimum of 304 patients/arm with ≥3 exacerbations in the previous year (608 total) will be randomized to achieve at least 90% power to detect a 30% reduction in benralizumab 100 mg versus placebo. This calculation assumes a 2-sided 5% alpha level, an annual placebo rate of 2, and a negative binomial dispersion parameter of 0.5.

A total of 868 patients are expected to be randomized in the study, also including a cohort of patients with 2 exacerbations in the previous year (approximately 260 patients) to characterize efficacy in patients with ≥2 exacerbations in the previous year.

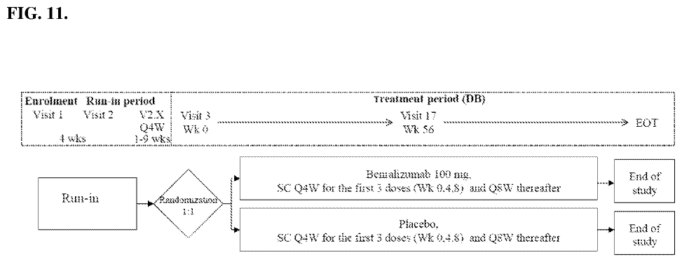

The general study design is summarized in FIG. 11.

The study population was identified based on the findings from GALATHEA and TERRANOVA, the previous Phase 3 studies of benralizumab in patients with moderate to very severe COPD. The two Phase 3 studies were of a nearly identical study design and included patients with a history of exacerbations despite double or triple background therapy with ICS/LABA, LAMA/LABA or ICS/LAMA/LABA across a range of baseline blood eosinophils. The primary population was patients with a baseline eosinophil count ≥220/μL in GALATHEA and TERRANOVA. In this new study, the primary population is patients with a baseline eosinophil count ≥300 eosinophils/μL who are on triple background therapy (ICS/LABA/LAMA) and with ≥3 exacerbations in the previous year. The overall population is patients with a baseline eosinophil count ≥300 eosinophils/μL who are on triple background therapy (ICS/LABA/LAMA) and with ≥2 exacerbations in the previous year.

In the primary analysis from GALATHEA and TERRANOVA, the benralizumab 100 mg dose resulted in a 17% and a 7% reduction over placebo in the primary endpoint of annual COPD exacerbation (moderate and severe combined) rate in GALATHEA and TERRANOVA, respectively. However, clinically relevant reductions in severe exacerbations (defined as exacerbations leading to hospitalization or death) were observed in both studies with the greatest effect observed in the benralizumab 100 mg group (43% and 32% for GALATHEA and TERRANOVA, respectively).

Pre-planned analyses evaluated specific subpopulations and resulted in the following:
Patients on triple (ICS/LABA/LAMA) background therapy (approximately 70% and 60% of patients in GALATHEA and TERRANOVA, respectively) had a consistent treatment effect on annual COPD exacerbation rate with benralizumab 100 mg in both studies: 18% and a 17% reduction over placebo, respectively.
Patients with more frequent exacerbations in the previous year (≥3 versus ≤2) had a greater treatment effect on annual COPD exacerbation rate and annual severe exacerbation rate with benralizumab 100 mg.
No treatment effect was observed on annual COPD exacerbation rate in patients with abaseline blood eosinophil count <220/μL.

Findings in these subpopulations were consistent across the studies and supported by pooled analyses. Taken together, these characteristics identify those patients most likely to benefit from benralizumab treatment.

An eosinophil cut point of ≥300/μL is used for this study. In the previous GALATHEA and TERRANOVA studies, no efficacy was seen in patients with low eosinophil counts (<220/μL). The treatment response was similar between patients with ≥220/μL and ≥300/μL. GOLD 2019 references a 300/μL threshold in terms of response to ICS and a 300/μL cutoff is more practical from a clinical perspective and aligns with clinical laboratory blood cell count data resolution.

A history of frequent exacerbations in the previous year (≥3) was the strongest and most consistent predictor of treatment response in GALATHEA and TERRANOVA. Therefore, this study will be powered for the primary population of patients with ≥3 exacerbations in the previous year. Study enrollment is expanded to also include patients with 2 exacerbations in the previous year to allow further exploration in a broader population (patients with ≥2 exacerbations in the previous year) given that there was evidence of a treatment effect in this subset of patients on triple (ICS/LABA/LAMA) background therapy with a baseline blood eosinophil count ≥220/μL in the GALATHEA and TERRANOVA studies. In a pooled post hoc analysis in this population (blood eosinophils ≥220/μL, ≥2 exacerbations in the previous year, on triple therapy), benralizumab 100 mg treatment resulted in a 20% reduction over placebo in the annual exacerbation rate and a 55% reduction over placebo in the annual severe exacerbation rate.

Chest CT imaging will be included in the study in order to provide needed information related to exclusion criterion 3 and to evaluate the potential effect of structural lung disease on efficacy.

### Inclusion Criteria

Patient (male or female) must be 40 to 85 years of age inclusive, at the time of signing the ICF.
Current smoker or ex-smoker with a tobacco history of ≥10 pack-years (1 pack year=20 cigarettes smoked per day for 1 year). (Note: electronic cigarette [e-cigarette] use does not contribute to the pack-year count for eligibility.)
History of moderate to very severe COPD with a post-bronchodilator FEV1/forced vital capacity (FVC)<0.70 and a post-bronchodilator FEV₁≤65% of predicted normal value at screening central spirometry assessment.
Documented history of 2 or more moderate and/or severe COPD exacerbations 1 that required treatment with systemic corticosteroids (at least 3 days or a single depot formulation injection) and/or hospitalization within 52 weeks prior to enrollment:
(a) Exacerbations treated with antibiotics alone are not considered as meeting the criterion unless it is accompanied by treatment with systemic corticosteroids and/or hospitalization.
(b) Hospitalization is defined as an inpatient admission ≥24 hours in the hospital, in an observation area, the emergency department, or other equivalent healthcare facility depending on the country and healthcare system.
(c) Previous exacerbations should be confirmed to have occurred while patient was on stable triple (ICS/LABA/LAMA) background therapy for COPD and not as a result of a step down in therapy, i.e. change from triple to dual therapy.
Documented use of triple (ICS/LABA/LAMA) background therapy for COPD throughout the year (52 weeks) prior to enrollment.
(a) ICS dose should be equivalent to ≥500 mcg of fluticasone propionate daily.
(b) Patient could have switched therapies during the previous year and/or stepped down for short periods of time, although the total cumulative duration that the patient was not using triple (ICS/LABA/LAMA) background therapy must not exceed 2 months.
(c) Patient must be on stable therapy/doses for the last 3 months prior to randomization. (individual component changes or switches between devices are allowed as long as the patient remains on ICS/LABA/LAMA with an acceptable ICS dose equivalent to ≥500 mcg of fluticasone propionate daily.)
Blood eosinophil count ≥300/μL at screening central laboratory testing, supported by at least 1 documented historical blood eosinophil count of ≥150/μL within 52 weeks of enrollment. In the absence of historical data, an additional blood eosinophil count may be obtained by repeating the testing during the run-in period (at least 4 weeks apart).
CAT total score ≥15 at Visit 1.
An exacerbation will be considered moderate if it required treatment with systemic corticosteroids and did not result in hospitalization. An exacerbation will be considered severe if it resulted in hospitalization.

### Exclusion Criteria

1 Clinically important pulmonary disease other than COPD (e.g. active lung infection, clinically significant bronchiectasis, pulmonary fibrosis, cystic fibrosis, hypoventilation syndrome associated with obesity, lung cancer, alpha 1 anti-trypsin deficiency, and primary ciliary dyskinesia).
2 Current diagnosis of asthma according to the Global Initiative for Asthma (GINA) or other accepted guidelines, prior history of asthma, or asthma-COPD overlap.
3 Radiological findings suggestive of a respiratory disease other than COPD that is contributing to the patient's respiratory symptoms. Radiological findings of solitary pulmonary nodules without appropriate follow up and demonstration of stability as per standard of care or findings suggestive of acute infection.
4 Another diagnosed pulmonary or systemic disease that is associated with elevated peripheral eosinophil counts (e.g. allergic bronchopulmonary aspergillosis/mycosis, eosinophilic granulomatosis with polyangitis, hypereosinophilic syndrome). Study treatment is defined as any investigational product (including marketed product comparator and placebo) or medical device intended to be administered to a patient according to the study protocol. Study treatment in this study refers to benralizumab or placebo.

### Concomitantt Therapy

All background COPD medications (e.g. ICS/LABA/LAMA, chronic antibiotics, etc.) and treatment given for COPD exacerbations in the 12 months prior to enrollment.
All other medications taken for any reason in the 3 months prior to enrollment
Concomitant treatments given during the study, with reason for the treatment (at each study visit)

COPD medications taken at the time of historical exacerbations, including any recent changes in these medications relative to the timing of the exacerbation, are to be captured in the eCRF.

The patient's usual triple (ICS/LABA/LAMA) background therapy formulation, dose, and regimen, and any other additional allowed COPD medications that may have been taken prior to enrollment, should be continued throughout the enrollment, run-in, and treatment period.

### Background (Maintenance) Medication

All patients are required to be treated with triple (ICS/LABA/LAMA) background therapy for COPD throughout the year prior to enrollment and during the course of the study. Patients must be on stable ICS/LABA/LAMA therapy/doses for the last 3 months prior to randomization with an acceptable ICS dose equivalent to ≥500 mcg of fluticasone propionate daily.

The aim of this study is to establish the treatment effect of benralizumab as an add-on to triple therapy. Therefore, the background medications should be maintained at the same dose and schedule from enrollment until the end of the study.

Minor changes of formulation of a patient's background inhaled medication, e.g. change of individual component (e.g. fluticasone to budesonide at equivalent dose) or switch between devices (e.g. budesonide/formoterol to fluticasone/salmeterol) are allowed as long as the patient remains on ICS/LABA/LAMA with an acceptable ICS dose. In case of such minor changes, the patient should be stable on the new treatment regimen for at least 4 weeks prior to randomization.

### Rescue Medication

Short-acting β2-agonists (SABAs, e.g. salbutamol, albuterol, terbutaline, levalbuterol) short-acting muscarinic antagonists (SAMA), SABA/SAMA combination or alternative rescue medication, as per local standard of care, may be used during the study in the event of worsening of COPD symptoms.

A list of allowed, restricted and prohibited medications are provided in Table 4.

TABLE 4

| | Allowed/Restricted/ | |
|---|---|---|
| Medication | Prohibited | Details |
| Background | Allowed | Patients should have evidence of having |
| (maintenance) | | been treated with triple |
| COPD medication: | | (ICS/LABA/LAMA) background therapy |
| triple | | for COPD throughout the year prior to Visit |
| (ICS/LABA/LAMA) | | 1 and have to be consistently treated with |
| | | COPD medications on acceptable doses* |
| | | throughout the study. |
| | | Triple background therapy components |
| | | may be used in fixed dose combination(s) |
| | | or separate devices. |
| | | Nebulized ICS (e.g. budesonide) and/or |
| | | LABA are accepted as part of maintenance |
| | | therapy. |
| | | *ICS component dose should be ≥500 mcg |
| | | fluticasone equivalent |
| SABA (short-acting β2- | Allowed as rescue | Prophylactic use of SABA in the absence of |
| agonists, e.g. | medication and for | symptoms is discouraged. However, if |
| salbutamol, | treatment of acute | deemed necessary by the patient |
| albuterol, terbutaline | COPD exacerbation | and investigator (e.g. prior to planned |
| levalbuterol) | | exercise), it can be used, but prophylactic |
| | | inhalations should not be recorded. |
| SAMA (short-acting | Allowed | SAMA taken at regularly scheduled |
| muscarinic antagonists = | | intervals (q 6-8 |
| short acting | | hours) will be considered equivalent to |
| anticholinergics; e.g. | | maintenance |
| ipratropium bromide, | | treatment with LAMA. |
| MDI or nebulized) | | Rescue use is allowed. |
| Systemic | Restricted | Allowed for treatment of COPD |
| corticosteroids | | exacerbation. |
| (tablets, suspension, or | | Allowed for treatment of an AE where there |
| injections) | | is no alternative treatment available, for the |
| | | duration of <4 weeks. |
| | | Disallowed within 4 weeks prior to |
| | | randomization and for |
| | | other reasons throughout the study. |
| Other | Restricted | Allowed for treatment of an AE where there |
| immunosuppressive | | is no alternative treatment available for the |
| medication (including | | duration of <4 weeks. |
| but | | Disallowed within 4 weeks prior to |
| not limited to: | | randomization and for |
| methotrexate, | | other reasons throughout the study. |
| troleandomycin, | | |
| cyclosporine, | | |
| azathioprine) | | |
| Antibiotics | Restricted | Allowed to treat COPD exacerbations |
| | | and/or AEs. |
| | | Chronic use (>3 wks) and use for the |
| | | prevention of COPD exacerbations is |
| | | disallowed, unless the patient is on stable |
| | | dose and regimen for ≥9 months prior to |
| | | randomization and has had ≥2 COPD |
| | | exacerbations while on stable |
| | | therapy. In all |
| | | other cases ≥6 weeks wash-out period |
| | | should be in place after the last dose of |
| | | antibiotic and prior to randomization. |
| Antitussives and | Allowed | |
| mucolytics | | |
| Dermal topical steroids, | Allowed | |
| nasal steroids, topical | | |
| ophthalmic and otic | | |
| corticosteroids | | |
| Antihistamines and | Allowed | |
| ephedrine containing | | |
| medications | | |
| Xanthines | Restricted | Allowed in a dose equivalent to |
| | | theophylline ≤400 mg q |
| | | day. For doses greater than 400 mg, the |
| | | dose has to be stable and blood levels |
| | | should be confirmed to be ≤12 mg/dL, |
| | | within 8 weeks prior to enrollment or |
| | | during screening. |
| | | Blood levels >12 mg/dL should be |
| | | managed according to local standards of |
| | | care. |

### Example 4—Study Assessments and Procedures

### Assessment of COPD Exacerbations:

For the purpose of the protocol, a COPD exacerbation will be defined as a worsening in the patient's usual COPD symptoms that is beyond normal day-to-day variation, is acute in onset, lasts 2 or more days (or 1 single day if the worsening is so rapid and profound that the treating physician judges that intensification of treatment cannot be delayed), and may warrant a change in regular medication and leads to any of the following:

Use of systemic corticosteroids for at least 3 days; a single depot injectable dose of corticosteroids will be considered equivalent to a 3-day course of systemic corticosteroids
Use of antibiotics
An inpatient hospitalization due to COPD (defined as an inpatient admission ≥24 hours in the hospital, an observation area, the emergency department, or other equivalent healthcare facility depending on the country and healthcare system)
Results in death

The worsening/onset of symptoms must include at least 1 major COPD symptom and at least one other major or minor symptom from the list below:
Major COPD symptoms: dyspnea, sputum volume, and sputum color
Minor COPD symptoms: cough, wheeze, sore throat, cold symptoms (rhinorrhea or nasal congestion), and fever without other cause

An exacerbation will be considered moderate if it requires treatment with systemic corticosteroids and/or antibiotics and does not result in hospitalization or death. An exacerbation will be considered severe if it results in hospitalization or death.

### Spirometry

Patients should be instructed not to use their ICS/LABA/LAMA medication within 12 hours (24 hours for once daily medications) of scheduled site visit spirometry as this will affect the pre-/post-bronchodilator FEV1 value: they may be taken subsequently, at the site. For the same reason, patients should not use their rescue (SABA and/or SAMA) medication within 6 hours of a scheduled site visit spirometry.

Multiple forced expiratory efforts (at least 3, but no more than 8) will be performed for each site spirometry session. Spirometry grading will conform to ATS/ERS acceptability and reproducibility criteria as detailed in the spirometry manual. The highest FEV1 and FVC will be based on the best efforts meeting acceptability criteria. The highest recorded FEV1 and FVC do not need to be from the same effort and can be derived from separate efforts. The absolute measurement (for FEV1 and FVC), and the percentage of predicted normal value (Quanjer et al 2012) will be recorded.

### Post-Bronchodilator Spirometry

Maximal bronchodilation will be induced using 4 inhalations of albuterol (90 μg metered dose) or salbutamol (100 μg metered dose) with or without a spacer device within 30±15 minutes of the final pre-bronchodilator spirometry measurement. Post-bronchodilator spirometry will be performed 20 to 30 minutes later. If a patient cannot tolerate 4 puffs of β agonist, a lower number of inhalations may be considered at the investigator's clinical judgment.

### Patient-Reported Outcomes

Daily: EXACT-PRO/E-RS: COPD (first 56 weeks only), background medication use, rescue medication use, and symptom severity questionnaire
Every 4 weeks for the first 56 weeks, and every 8 weeks thereafter: SGRQ, CAT, and EQ-5D-5L
week after each scheduled visit for IP administration: Onset of Effect Questionnaire (OEQ)-Question 2 (first 56 weeks only)
   St. George's Respiratory Questionnaire (SGRQ)

The SGRQ is a 50-item PRO instrument developed to measure the health status of patients with airway obstruction diseases (Jones et al 1991). The questionnaire is divided into two parts: part 1 consists of 8 items pertaining to the severity of respiratory symptoms in the preceding 4 weeks: part 2 consists of 42 items related to the daily activity and psychosocial impacts of the individual's respiratory condition. The SGRQ yields a total score and 3 domain scores (symptoms, activity, and impacts). The total score indicates the impact of disease on overall health status. This total score is expressed as a percentage of overall impairment, in which 100 represents the worst possible health status and 0 indicates the best possible health status. Likewise, the domain scores range from 0 to 100, with higher scores indicative of greater impairment. Specific details on the scoring algorithms are provided by the developer in a user manual (Jones 2009).

The patient will complete the SGRQ on the eDiary at the site for Visit 1 screening) and Visit 3 (randomization) prior to other study procedures. During the run-in period and up to Week 56 of the treatment period, the SGRQ will be available every 28±7 days, to be completed preferably at home prior to the study visit or at the site during the scheduled visit. After Week 56, the SGRQ will be available every 56±7 days, to be completed preferably at home prior to the study visit or at the site during the scheduled visit.

### COPD Assessment Test (CAT)

The CAT is an 8-item PRO developed to measure the impact of COPD on health status (Jones et al 2009). The instrument uses semantic differential 6-point response scales which are defined by contrasting adjectives to capture the impact of COPD. Content includes items related to cough, phlegm, chest tightness, breathlessness going up hills/stairs, activity limitation at home, confidence leaving home, sleep, and energy. A CAT total score is the sum of item responses. Scores range from 0 to 40 with higher scores indicative of greater COPD impact on health status.

The patient will complete the CAT at the site for Visit 1 (screening) and Visit 3 (randomization) prior to other study procedures. During the run-in period and up to Week 56 of the treatment period, the CAT will be available every 28±7 days, to be completed preferably at home prior to the study visit or at the site during the scheduled visit. After Week 56, the CAT will be available every 56±7 days, to be completed preferably at home prior to the study visit or at the site during the scheduled visit.

### European Quality of Life-5 Dimensions (EQ-5D-5L)

The EQ-5D-5L questionnaire assesses 5 dimensions: mobility, self-care, usual activities, pain/discomfort and anxiety/depression. Each dimension has 5 response options (no problems, slight problems, moderate problems, severe problems, and extreme problems) that reflect increasing levels of difficulty.

The patient will be asked to indicate his/her current health state by selecting the most appropriate level in each of the 5 dimensions. The questionnaire also includes a visual analog scale (VAS), where the patient will be asked to rate current health status on a scale of 0 to 100, with 0 being the worst imaginable health state. The patient will complete the EQ-5D-5L on the eDiary at the site for Visit 1 (screening) and Visit 3 (randomization) prior to other study procedures. During the run-in period and up to Week 56 of the treatment period, the EQ-5D-5L will be available every 28±7 days, to be completed preferably at home prior to the study visit or at the site during the scheduled visit. After Week 56, the EQ-5D-5L will be available every 56±7 days, to be completed preferably at home prior to the study visit or at the site during the scheduled visit.

### Statistical Considerations

The primary efficacy endpoint is the annualized rate of moderate and severe COPD exacerbations over the first 56 weeks. The primary endpoint will be assessed first in the primary population (patients with ≥3 exacerbations in the previous year) and then assessed on the overall population of patients with ≥2 exacerbations in the previous year. A treatment policy estimand will be applied to the primary analysis of the primary endpoint whereby all data is included, regardless of whether a patient remains on blinded IP or not.

The null hypothesis is that the exacerbation rate on benralizumab 100 mg is equal to the exacerbation rate on placebo. The alternative hypothesis is that the exacerbation rate on benralizumab 100 mg is not equal to the exacerbation rate on placebo, i.e.:
H₀: Rate ratio (benralizumab 100 mg vs. Placebo)=1
H₁: Rate ratio (benralizumab 100 mg vs. Placebo)≠1

Hypothesis testing for the primary analysis, annualized rate of moderate and severe COPD exacerbations over 56 weeks in patients with ≥3 exacerbations in the previous year, will be performed at the 2-sided 5% significance level. If the p-value is less than 0.05 and the treatment effect favors benralizumab, reject H0 and accept H₁.

### Sample Size Determination

The study will recruit patients on triple (ICS/LABA/LAMA) background therapy with baseline eosinophil counts ≥300/μL and ≥2 exacerbations in the previous year, with at least 70% of patients with ≥3 exacerbations in the previous year. This study is powered for the primary population of patients with ≥3 exacerbations in the previous year. For the primary endpoint of annualized exacerbation rate over the first 56 weeks, a minimum of 304 patients/arm with ≥3 exacerbations in the previous year (608 total) will be randomized to achieve at least 90% power to detect a 30% reduction in benralizumab 100 mg versus placebo. This calculation assumes a 2-sided 5% alpha level, an annual placebo rate of 2, and a negative binomial dispersion parameter of 0.5.

A total of 868 patients are expected to be randomized in the study, also including a cohort of patients with 2 exacerbations in the previous year to characterize efficacy in patients with ≥2 exacerbations in the previous year.

For the key secondary endpoint of annualized severe exacerbation rate over the entire treatment period through EOT, there is expected to be at least 80% power to detect a 40% reduction in benralizumab 100 mg versus placebo in patients with ≥3 exacerbations in the previous year and at least 90% power in patients with ≥2 exacerbations in the previous year. This calculation assumes placebo rates of 0.30 and 0.27 in the primary and overall populations respectively, a negative binomial dispersion parameter of 2.7, and mean follow-up time of 2 years. A 2-sided 5% significance level in the >3 and >2 prior exacerbation groups is assumed following the multiple testing procedure.

### Efficacy Endpoints

All efficacy endpoints (listed in Table 5) will be analyzed in patients with a history of ≥3 exacerbations in the previous year (primary population) and repeated in patients with a history of ≥2 exacerbations in the previous year (overall population). Where serially measured endpoints (e.g. SGRQ and FEV1) are also collected beyond Week 56, additional models similar to those fitted on the data up to Week 56 will be used to assess these endpoints over the longer follow-up period through EOT.

TABLE 5
Efficacy Endpoints

| Category | Endpoint | Main timepoint for analysis |
|---|---|---|
| Primary | Annualized rate of moderate | Over first 56 weeks |
| | and severe COPF | |
| | exacerbations | |
| Key | Annualized rate of severe | Through EOT |
| secondary | COPD exacerbations | |
| | Change from baseline in | Week 56 |
| | SGRQ total score | |
| | Change from baseline in pre- | Week 56 |
| | dose/pre-bronchodilator | |
| | FEV1 | |
| Secondary | Annualized rate of COPD | Through EOT |
| | exacerbations that are | |
| | associated with an emergency | |
| | room visit or a hospitalization | |
| | Time to the first COPD | During first 56 weeks |
| | exacerbation | |
| | Change from baseline in | Week 56 |
| | SGRQ domain scores | |
| | Change from baseline in CAT | Week 56 |
| | total score | |
| | E-RS: COPD total and | Week 56 |
| | domain scores | |
| | All cause and respiratory- | Through EOT |
| | related mortality rate | |
| | Annual rate of | Through EOT |
| | hospitalizations due to | |
| | COPD; Length of hospital | |
| | stay; ICU days; annual rate of | |
| | hospitalizations and | |
| | emergency | |
| | department visits combined | |
| | due to COPD; annual rate of | |
| | unscheduled outpatient visits | |
| | including unscheduled visits | |
| | to study | |
| | sites due to COPD; and | |
| | annual rate of unscheduled | |
| | healthcare | |
| | encounters due to COPD | |

### Primary Analysis Method

The primary efficacy variable is the annualized rate of moderate and severe COPD exacerbations over the first 56 weeks, and the primary analysis is to compare the annualized COPD exacerbation rate of benralizumab 100 mg with placebo in patients with a history of ≥3 exacerbations in the previous year.

Exacerbation rate in the benralizumab treatment group will be compared to exacerbation rate in the placebo group using a negative binomial model. The response variable in the model will be the number of COPD exacerbations over the first 56 weeks of the treatment period. The model will include covariates of treatment group, region, and the number of exacerbations in the previous year. The logarithm of the follow-up time during the first 56 weeks will be used as an offset variable in the model.

The estimated treatment effect (i.e. the rate ratio of benralizumab versus placebo), corresponding 95% confidence interval (CI), and 2-sided p-value for the rate ratio will be presented. In addition, the exacerbation rate and the corresponding 95% CI within each treatment group will be presented.

### Analysis Methods for Secondary Efficacy Variables

The primary analysis will be repeated in patients with a history of ≥2 exacerbations in the previous year. All secondary efficacy endpoints will be analyzed in patients with a history of ≥3 exacerbations in the previous year and repeated in patients with a history of ≥2 exacerbations in the previous year. Where serially measured endpoints (e.g. SGRQ and FEV1) are also collected beyond Week 56, additional models similar to those fitted on the data up to Week 56 will be used to assess these endpoints over the longer follow-up period through EOT.

Annualized rate of COPD exacerbations leading to hospitalization or death (i.e. severe exacerbations) will be analyzed through EOT using a similar negative binomial model as outlined above. The annualized rate of COPD exacerbations associated with an emergency room visit or a hospitalization through EOT will be analyzed similarly.

The proportion of patients with ≥1 COPD exacerbation will be addressed as a supportive variable to the primary objective. The proportion in the benralizumab group will be compared with the proportion in the placebo group using a logistic regression model with region and number of exacerbations in the previous year as covariates.

Time to first COPD exacerbation will be analyzed as another supportive efficacy variable to the primary objective to explore the extent to which treatment with benralizumab delays the time to first exacerbation compared with placebo. A Cox proportional hazard model will be fitted to data with the covariates of treatment, region, and number of exacerbations in the previous year.

Change from baseline in SGRQ total score and three domain scores (symptoms, activity, and impacts) at Week 56 will be compared between benralizumab and placebo using a repeated measures analysis in the FAS. The dependent variable will be the change from baseline in SGRQ score at post-baseline protocol-specified visits (up to the Week 56 visit). Treatment group will be fitted as the explanatory variable, and region, visit, the interaction between visit and treatment, and baseline SGRQ score will be fitted as covariates. Visit will be fitted as a categorical variable, and the variance-covariance matrix will be assumed in the following order: unstructured, Toeplitz, first-order autoregressive, compound symmetric, and variance components. If the procedure does not converge, the next covariance matrix will be used. The model is:

Change in SGRO score=Treatment group+baseline SGRO score+region+visit+treatment*visit

The proportion of patients with ≥4-point decrease (improvement) in SGRQ total score at Week 56 in the benralizumab group will be compared with the proportion in the placebo group using a Cochran-Mantel-Haenszel test controlling for region.

Change from baseline in pre-dose/pre-bronchodilator FEV1 at Week 56 will be analyzed in patients with a baseline pre-dose/pre-bronchodilator FEV1 and at least one post-randomization pre-dose/pre-bronchodilator FEV1 using a similar repeated measures analysis model as the model for change from baseline in SGRQ score.

Change from baseline in CAT total score at Week 56 and E-RS: COPD total score and domain scores at Week 56 will be analyzed separately using a similar model to the model for change from baseline in SGRQ score.

Those skilled in the art will recognize, or be able to ascertain using no more than routine experimentation, many equivalents to the specific aspects of the disclosure described herein. Such equivalents are intended to be encompassed by the following claims.

Various publications are cited herein, the disclosures of which are incorporated by reference in their entireties.

Although the foregoing invention has been described in some detail by way of illustration and example for purposes of clarity of understanding, it will be obvious that certain changes and modifications can be practiced within the scope of the appended claims.

SEQUENCE LISTING
>US20100291073_1 Sequence 1 from Patent US
20100291073 Organism: Homo sapiens
SEQ ID NO: 1
DIQMTQSPSSLSASVGDRVTITCGTSEDIINYLNWYQQKPGKAPKLLIYH
TSRLQSGVPSRFSGSGSGTDFTLTISSLQPEDFATYYCQQGYTLPYTFGQ
GTKVEIK
>US20100291073_2 Sequence 2 from Patent US
20100291073 Organism: Homo sapiens
SEQ ID NO: 2
DIQMTQSPSSLSASVGDRVTITCGTSEDIINYLNWYQQKPGKAPKLLIYH
TSRLQSGVPSRFSGSGSGTDFTLTISSLQPEDFATYYCQQGYTLPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
>US20100291073_3 Sequence 3 from Patent US
20100291073 Organism: Homo sapiens
SEQ ID NO: 3
EVQLVQSGAEVKKPGASVKVSCKASGYTFTSYVIHWVRQRPGQGLAWMGY
INPYNDGTKYNERFKGKVTITSDRSTSTVYMELSSLRSEDTAVYLCGREG
IRYYGLLGDYWGQGTLVTVSS
>US20100291073_4 Sequence 4 from Patent US
20100291073 Organism: Homo sapiens
SEQ ID NO: 4
EVQLVQSGAEVKKPGASVKVSCKASGYTFTSYVIHWVRQRPGQGLAWMGY
INPYNDGTKYNERFKGKVTITSDRSTSTVYMELSSLRSEDTAVYLCGREG
IRYYGLLGDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K
>US20100291073_5 Sequence 5 from Patent US
20100291073 Organism: Homo sapiens
SEQ ID NO: 5
DLLPDEKISLLPPVNFTIKVTGLAQVLLQWKPNPDQEQRNVNLEYQVKIN
APKEDDYETRITESKCVTILHKGFSASVRTILQNDHSLLASSWASAELHA
PPGSPGTSIVNLTCTTNTTEDNYSRLRSYQVSLHCTWLVGTDAPEDTQYF
LYYRYGSWTEECQEYSKDTLGRNIACWFPRTFILSKGRDWLAVLVNGSSK
HSAIRPFDQLFALHAIDQINPPLNVTAEIEGTRLSIQWEKPVSAFPIHCF
DYEVKIHNTRNGYLQIEKLMTNAFISIIDDLSKYDVQVRAAVSSMCREAG
LWSEWSQPIYVGNDEHKPLREWFVIVIMATICFILLILSLICKICHLWIK
LFPPIPAPKSNIKDLFVTTNYEKAGSSETEIEVICYIEKPGVETLEDSVF
>US20100291073_6 Sequence 6 from Patent US
20100291073 Organism: Mus musculus
SEQ ID NO: 6
DLLNHKKFLLLPPVNFTIKATGLAQVLLHWDPNPDQEQRHVDLEYHVKIN
APQEDEYDTRKTESKCVTPLHEGFAASVRTILKSSHTTLASSWVSAELKA
PPGSPGTSVTNLTCTTHTVVSSHTHLRPYQVSLRCTWLVGKDAPEDTQYF
LYYRFGVLTEKCQEYSRDALNRNTACWFPRTFINSKGFEQLAVHINGSSK
RAAIKPFDQLFSPLAIDQVNPPRNVTVEIESNSLYIQWEKPLSAFPDHCF
NYELKIYNTKNGHIQKEKLIANKFISKIDDVSTYSIQVRAAVSSPCRMPG
RWGEWSQPIYVGKERKSLVEWHLIVLPTAACFVLLIFSLICRVCHLWTRL
FPPVPAPKSNIKDLPWTEYEKPSNETKIEVVHCVEEVGFEVMGNSTF
VH CDR1
SEQ ID NO: 7
SYVIH
VH CDR2
SEQ ID NO: 8
YINPYNDGTKYNERFKG
VH CDR3
SEQ ID NO: 9
EGIRYYGLLGDY
VL CDR1
SEQ ID NO: 10
GTSEDIINYLN
VL CDR2
SEQ ID NO: 11
HTSRLQS
VL CDR3
SEQ ID NO: 12
QQGYTLPYT

What is claimed is:

1. A method of treating chronic obstructive pulmonary disease (COPD) in a human COPD patient, comprising administering to the patient a dose of 100 mg of benralizumab or an antigen-binding fragment thereof, wherein prior to administration, the patient:
(a) has a blood eosinophil count of ≥300 eosinophils/μL;
(b) has experienced ≥2 prior exacerbations in the year prior to administration; and
(c) is on triple background therapy comprising inhaled corticosteroid (ICS), long-acting beta agonist (LABA), and long-acting muscarinic antagonist (LAMA).

2. A method of reducing the annual exacerbation rate of chronic obstructive pulmonary disease (COPD) comprising administering to a human COPD patient a dose of 100 mg of benralizumab or an antigen-binding fragment thereof, wherein prior to administration, the patient:
(a) has a blood eosinophil count of ≥300 eosinophils/μL;
(b) has experienced ≥2 prior exacerbations in the year prior to administration; and
(c) is on triple background therapy comprising inhaled corticosteroid (ICS), long-acting beta agonist (LABA), and long-acting muscarinic antagonist (LAMA).

3. A method of increasing forced expiratory volume in one second (FEV₁) in a human chronic obstructive pulmonary disease (COPD) patient comprising administering to the patient a dose of 100 mg of benralizumab or an antigen-binding fragment thereof, wherein prior to administration, the patient:
(a) has a blood eosinophil count of ≥300 eosinophils/μL;
(b) has experienced ≥2 prior exacerbations in the year prior to administration; and
(c) is on triple background therapy comprising inhaled corticosteroid (ICS), long-acting beta agonist (LABA), and long-acting muscarinic antagonist (LAMA).

4. A method of increasing forced vital capacity (FVC) in a human chronic obstructive pulmonary disease (COPD) patient comprising administering to the patient a dose of 100 mg of benralizumab or an antigen-binding fragment thereof, wherein prior to administration, the patient:
(a) has a blood eosinophil count of ≥300 eosinophils/μL;
(b) has experienced ≥2 prior exacerbations in the year prior to administration; and
(c) is on triple background therapy comprising inhaled corticosteroid (ICS), long-acting beta agonist (LABA), and long-acting muscarinic antagonist (LAMA).

5. The method of any one of claims 1-4, wherein a COPD questionnaire score assessing COPD symptoms in the human COPD patient is improved.

6. The method of any one of 1-5, wherein the patient has severe or very severe COPD as defined by Global Initiative for Chronic Obstructive Lung Disease (GOLD).

7. The method of claim 6, wherein the patient has very severe COPD as defined by GOLD.

8. The method of any one of claims 1-7, wherein the administration reduces the exacerbation rate of COPD.

9. The method of any one of claims 2-8 wherein the exacerbation rate is reduced by at least 20%.

10. The method of any one of claims 2-8 wherein the exacerbation rate is reduced by at least 30%.

11. The method of any one of claims 2-8 wherein the exacerbation rate is reduced by at least 40%.

12. The method of any one of claims 2-8 wherein the exacerbation rate is reduced by at least 50%.

13. The method of any one of claims 2-8 wherein the exacerbation rate is reduced by at least 60%.

14. The method of any one of claims 1-13, wherein the administration increases the patient's FEV₁.

15. The method of any one of claims 1-13, wherein the administration increases the patient's FVC.

16. The method of claim 6, wherein the COPD questionnaire is the COPD-Specific Saint George's Respiratory Questionnaire (SGRQ-C).

17. The method of claim 16, wherein the patient's SGRQ-C (symptom) score decreases by at least 5.

18. The method of any one of claims 1-17, where the patient's SGRQ-C (symptom) score decreases by at least 9.

19. The method of any one of claims 1-17, wherein the patient has experienced ≥3 prior exacerbations in the year prior to the administration.

20. The method of any one of claims 1-19, wherein the patient receiving benralizumab is concurrently being treated with triple background therapy, wherein triple background therapy comprises inhaled corticosteroid (ICS), long-acting beta agonist (LABA), and long-acting muscarinic antagonist (LAMA).

21. The method of any one of claims 1-19, wherein the patient receiving benralizumab is not concurrently being treated with triple background therapy, wherein triple background therapy comprises inhaled corticosteroid (ICS), long-acting beta agonist (LABA), and long-acting muscarinic antagonist (LAMA).

22. The method of any one of claims 1-21, wherein the patient had an FEV₁ of ≤80% predicted value prior to the administration of benralizumab or an antigen-binding fragment thereof.

23. The method of any one of claims 1-21, wherein the patient had an FEV₁ of ≤70% predicted value prior to the administration of benralizumab or an antigen-binding fragment thereof.

24. The method of any one of claims 1-21, wherein the patient had an FEV₁ of ≤65% predicted value prior to the administration of benralizumab or an antigen-binding fragment thereof.

25. The method of any one of claims 1-21, wherein the patient had an FEV₁/forced vital capacity (FVC) of <0.70 prior to the administration of benralizumab or an antigen-binding fragment thereof.

26. The method of any one of claims 1-25, wherein at least two doses of benralizumab or an antigen-binding fragment thereof are administered.

27. The method of claim 26, wherein benralizumab or antigen-binding fragment thereof is administered once every four weeks to once every twelve weeks.

28. The method of any one of claims 1-26, wherein benralizumab or an antigen-binding fragment thereof is administered once every four weeks.

29. The method of any one of claims 1-26, wherein benralizumab or an antigen-binding fragment thereof is administered once every eight weeks.

30. The method of any one of claims 1-26, wherein benralizumab or antigen-binding fragment thereof is administered once every four weeks for twelve weeks and then once every eight weeks.

31. The method of any one of claims 1-30, wherein the administration is subcutaneous.

32. A method of treating chronic obstructive pulmonary disease (COPD) in a human COPD patient, comprising administering to the patient a dose of 100 mg benralizumab or an antigen-binding fragment thereof, wherein prior to administration, the patient:
(a) has a blood eosinophil count of ≥300 eosinophils/μl;
(b) has experienced ≥3 prior exacerbations in the year prior to administration; and
(c) is on triple background therapy comprising inhaled corticosteroid (ICS), long-acting beta agonist (LABA), and long-acting muscarinic antagonist (LAMA).

33. A method of reducing the exacerbation rate of chronic obstructive pulmonary disease (COPD) in a human COPD patient comprising administering to the patient a dose of 100 mg benralizumab or an antigen-binding fragment thereof, wherein prior to administration, the patient:
(a) has a blood eosinophil count of ≥300 eosinophils/μL;
(b) has experienced ≥3 prior exacerbations in the year prior to administration; and
(c) is on triple background therapy comprising inhaled corticosteroid (ICS), long-acting beta agonist (LABA), and long-acting muscarinic antagonist (LAMA).

34. A method of increasing forced expiratory volume in one second (FEV₁) in a human chronic obstructive pulmonary disease (COPD) patient comprising to the patient a dose of 100 mg benralizumab or an antigen-binding fragment thereof, wherein prior to administration, the patient:
(a) has a blood eosinophil count of ≥300 eosinophils/μL;
(b) has experienced ≥3 prior exacerbations in the year prior to administration; and
(c) is on triple background therapy comprising inhaled corticosteroid (ICS), long-acting beta agonist (LABA), and long-acting muscarinic antagonist (LAMA).

35. A method of increasing forced vital capacity (FVC) in a human chronic obstructive pulmonary disease (COPD) patient comprising administering to the patient a dose of 100 mg benralizumab or an antigen-binding fragment thereof, wherein prior to administration, the patient:
(a) has a blood eosinophil count of ≥300 eosinophils/μL;
(b) has experienced ≥3 prior exacerbations in the year prior to adminstration; and
(c) is on triple background therapy comprising inhaled corticosteroid (ICS), long-acting beta agonist (LABA), and long-acting muscarinic antagonist (LAMA).

36. The method of any one of claims 32-25, wherein the patient receiving benralizumab is concurrently being treated with triple background therapy, wherein triple background therapy comprises inhaled corticosteroid (ICS), long-acting beta agonist (LABA), and long-acting muscarinic antagonist (LAMA).

37. The method of any one of claims 32-35, wherein the patient receiving benralizumab is not concurrently being treated with triple background therapy, wherein triple background therapy comprises inhaled corticosteroid (ICS), long-acting beta agonist (LABA), and long-acting muscarinic antagonist (LAMA).

38. A method of treating chronic obstructive pulmonary disease (COPD) in a human COPD patient, comprising administering to the patient a dose of 100 mg of benralizumab or an antigen-binding fragment thereof, wherein prior to first administration, the patient:
(a) has a blood eosinophil count of ≥300 eosinophils/μL;
(b) has experienced ≥2 prior exacerbations in the year prior to administration; and
(c) is on triple background therapy comprising inhaled corticosteroid (ICS), long-acting beta agonist (LABA), and long-acting muscarinic antagonist (LAMA); and wherein the patient no longer receives triple background therapy after administering 100 mg of benralizumab once every four weeks for twelve weeks and then once every eight weeks.

39. A method of treating chronic obstructive pulmonary disease (COPD) in a human COPD patient, comprising administering to the patient a dose of 100 mg of benralizumab or an antigen-binding fragment thereof, wherein prior to first administration, the patient:
(a) has a blood eosinophil count of ≥300 eosinophils/μL;
(b) has experienced ≥3 prior exacerbations in the year prior to administration; and
(c) is on triple background therapy comprising inhaled corticosteroid (ICS), long-acting beta agonist (LABA), and long-acting muscarinic antagonist (LAMA); and wherein the patient no longer receives triple background therapy after administering 100 mg of benralizumab once every four weeks for twelve weeks and then once every eight weeks.
